(19) United States
(12) Patent Application Publication
(10) Pub. No.: US 2016/0374738 A1
(43) Pub. Date: Dec. 29, 2016

(54) Combined Intramedullary and Extramedullary Surgical Aiming System And Method

(71) Applicant: Interfix, LLC, Houston, TX (US)

(72) Inventors: Joel Smith, San Diego, CA (US); Aaron Osborne, Redding, CA (US); Brian Chalkin, Tulsa, OK (US)

(21) Appl. No.: 14/749,320

(22) Filed: Jun. 24, 2015

(51) Int. Cl. A61B 17/72; A61B 17/88; A61B 17/80; A61B 17/17

(52) U.S. Cl. CPC A61B 17/725; A61B 17/1725; A61B 17/1728; A61B 17/88; A61B 17/8057; A61B 17/80; A61B 2017/90

(57) ABSTRACT

A surgical aiming system is disclosed comprising a one or more fixation plates, an aiming assembly, a one or more nails and a plurality of screws. Said aiming assembly comprising a channeling assembly and a plurality of channels. Said one or more fixation plates comprising a plurality of plate apertures capable of receiving a portion of said plurality of screws. Said plurality of channels in said aiming assembly are capable of aligning a portion of said plurality of screws with said plurality of plate apertures in a first fixation plate. Said plurality of channels receive and align said plurality of screws as they are secured to said first bone. Said first fixation plate being extramedullary attached to a portion of said surface of said first bone; and said plurality of channels of said aiming assembly being aligned to identify a one or more intramedullary locations within said first bone.

### CROSS-REFERENCE TO RELATED APPLICATIONS

This application is a continuation in part of and claims benefit to U.S. patent application Ser. No. 14/306,683 (filed 2014 Jun. 17) which is currently under a notice of allowance (hereafter the “Parent Application”). Said Parent Application was the nonprovisional filing made on the basis of US Provisional Patent Application Numbers 61890240 (filed on Oct. 12, 2013), and 61934692 (filed Jan. 31, 2014). This application hereby incorporates all of these applications by reference.

### STATEMENT REGARDING FEDERALLY SPONSORED RESEARCH OR DEVELOPMENT (IF APPLICABLE)

Not applicable.

### REFERENCE TO SEQUENCE LISTING, A TABLE, OR A COMPUTER PROGRAM LISTING COMPACT DISC APPENDIX (IF APPLICABLE)

Not applicable.

### BACKGROUND OF THE INVENTION

This disclosure relates generally to a surgical aiming system and method. Surgical systems for aiming plates and screws are well-known and include products by companies such as Synthes®, Zimmer®, Stryker®, and Biomet®. A few examples of surgical systems includes: U.S. Pat. No. 4,465,065A, US20110224736A1, U.S. Pat. No. 8,523,862B2, U.S. Pat. No. 8,523,919B2, U.S. Pat. No. 6,926,720B2, US20090228047A1, U.S. Pat. No. 8,556,945B2, U.S. Pat. No. 8,545,540B2, U.S. Pat. No. 6,342,057B1, U.S. Pat. No. 4,465,065, U.S. Pat. No. 5,366,457, U.S. Pat. No. 5,458,654, U.S. Pat. No. 5,658,283, U.S. Pat. No. 5,853,415, U.S. Pat. No. 5,928,234, U.S. Pat. No. 6,514,253, U.S. Pat. No. 6,527,775, U.S. Pat. No. 6,579,293, U.S. Pat. No. 6,692,496, U.S. Pat. No. 6,706,046, U.S. Pat. No. 6,746,453, US20030055428, US20030216742, U.S. Pat. No. 7,488,323, U.S. Pat. No. 7,588,577, U.S. Pat. No. 7,621,920, U.S. Pat. No. 7,648,508, U.S. Pat. No. 7,686,808, U.S. Pat. No. 7,727,236, U.S. Pat. No. 7,771,441, U.S. Pat. No. 7,833,230, U.S. Pat. No. 7,837,689, U.S. Pat. No. 8,034,056, U.S. Pat. No. 8,114,093, U.S. Pat. No. 8,142,432, U.S. Pat. No. 8,162,950, U.S. Pat. No. 8,282,638, U.S. Pat. No. 8,439,932, U.S. Pat. No. 8,679,130, US20110106086, WO2007086854A1, WO2007109437A2, WO2008098016A2, and WO2009121144A1.

However, none of the prior inventions and/or patents, taken either singularly or in combination, is seen to describe the instant disclosure as claimed. Accordingly, an improved surgical aiming system and method would be advantageous.

### BRIEF SUMMARY OF THE INVENTION

A surgical aiming system is disclosed comprising a one or more fixation plates, an aiming assembly, a one or more nails and a plurality of screws. Said one or more fixation plates comprising a first fixation plate. Said aiming assembly comprising a channeling assembly and a plurality of channels. Said one or more fixation plates comprising a plurality of plate apertures capable of receiving a portion of said plurality of screws. Said plurality of channels in said aiming assembly are capable of aligning a portion of said plurality of screws with said plurality of plate apertures in said first fixation plate. Said first fixation plate having a first end, a second end, a top surface, and a bottom surface. Said plurality of plate apertures comprising a first plate aperture and a second first plate aperture. Said channeling assembly having a thickness, a top surface, and a bottom surface. Said bottom surface of said first fixation plate substantially couples with a surface of a first bone of a skeletal system. Said bottom surface said aiming assembly substantially couples with said top surface of said first fixation plate. Said plurality of channels receive and align said plurality of screws as said plurality of screws are inserted through said plurality of plate apertures and secured to said first bone. Said first fixation plate being extramedullary attached to a portion of said surface of said first bone; and said plurality of channels of said aiming assembly being aligned to identify a one or more intramedullary locations including an entry point for said one or more nails, and an alignment and entry point for each among said plurality of plate apertures.

### BRIEF DESCRIPTION OF THE SEVERAL VIEWS OF THE DRAWING

FIGS. 1A and 1B illustrate an elevated front view of a skeletal system and a perspective anterior view of a first bone with a first fixation plate.

FIGS. 2A, 2B and 2C illustrate a perspective overview, an elevated bottom view and an elevated side view of said first fixation plate.

FIGS. 3A and 3B illustrate an elevated cross-section view of said central plate aperture and said fourth plate aperture, respectively.

FIGS. 4A, 4B and 4C illustrate a perspective overview of a drill guide with a screw, with said drill guide, and said screw inserted into a wireframe view of said first bone.

FIGS. 5A and 5B illustrate a perspective overview of said first fixation plate attached to said first bone with a plurality of screws.

FIG. 5A illustrates said first bone as a solid view and 5B illustrates said first bone in a wireframe view.

FIGS. 5C and 5D illustrate a cross-section elevated front view of said first fixation plate and said first bone.

FIGS. 6A and 6B illustrate an exploded perspective first side overview and second side overview of said surgical aiming system with said first bone.

FIGS. 7A, 7B and 7C illustrate two exploded views and an attached perspective overview of said intramedullary rod guide, said channeling assembly, said first fixation plate and said first bone.

FIGS. 8A and 8B illustrate an exploded perspective front view and overview of said triangulating screw guide, said thumb screw and said first intramedullary nail.

FIG. 9 illustrates a wireframe perspective overview said first intramedullary nail inserted into said first bone.

FIGS. 10A and 10B illustrate a perspective lower view and overview of said triangulating screw guide, said thumb screw and said first intramedullary nail attached to one another with said first bone and channeling assembly in a wireframe view.

FIGS. 11A and 11B illustrate a perspective overview and a perspective lower view of said triangulating screw guide.

FIGS. 12A and 12B illustrate a perspective side view and a perspective front view of said first intramedullary nail attached to said triangulating screw guide with three of said plurality of screws.

FIGS. 13A, 13B, 13C, 13D and 13E illustrate a series of perspective overviews of a stacked drilling system.

FIGS. 14A and 14B illustrate a wireframe view and a solid view of a perspective overview of said first bone comprising said first intramedullary nail inserted and attached to said first fixation plate with said channeling assembly, said triangulating screw guide, and said thumb screw.

FIG. 15 illustrates a perspective overview of said first bone 100 with said first fixation plate attached, said first intramedullary nail inserted, and a portion of said plurality of screws installed.

FIGS. 16A, 16B and 16C illustrate a perspective overview said surgical aiming system attached to said first bone with said first fixation plate and a second fixation plate.

FIGS. 17A, 17B, 17C and 17D illustrate an elevated side view, an elevated top view, an elevated front view and a perspective lower view of a triangulating screw guide.

FIGS. 18A and 18B illustrate a series of perspective overviews of said triangulating screw guide aligned with said first fixation plate and said second fixation plate.

FIGS. 19A and 19B illustrate a perspective wireframe overview and detailed view of said triangulating screw guide, second fixation plate, a one or more nails, and said first fixation plate attached to said first bone.

FIGS. 20A and 20B illustrate a perspective overview of a first aiming assembly, said first fixation plate and said first bone.

FIGS. 21A and 21B illustrate a perspective overview of a second aiming assembly, said first bone and said first fixation plate.

FIGS. 22A and 22B illustrate a perspective overview of a first channeling assembly, said first fixation plate and said first bone.

FIGS. 23A, 23B and 23C illustrate a perspective side view of said surgical aiming system.

FIG. 23A includes said first bone 100 and FIGS. 23B-23C do not include said first bone.

FIGS. 24A and 24B illustrate a perspective lower view of said surgical aiming system without said first bone.

FIG. 24A includes said plurality of screws and FIG. 24B does not.

FIGS. 25A and 25B illustrate perspective lower view and overview of said triangulating screw guide, said thumb screw and an alternative nail attached to one another with said first bone and channeling assembly in a wireframe view.

FIGS. 26A and 26B illustrate a perspective lower view of said surgical aiming system without said first bone.

FIG. 26A includes said plurality of screws and FIG. 26B does not.

### DETAILED DESCRIPTION OF THE INVENTION

Described herein is a combined intramedullary and extramedullary surgical aiming system and method. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

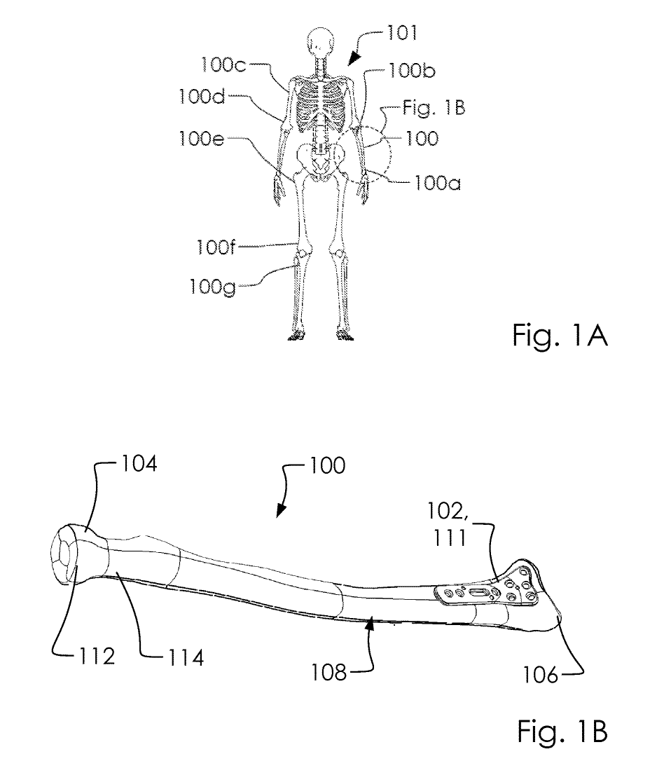

FIGS. 1A and 1B illustrate an elevated front view of a skeletal system 101 and a perspective anterior view of a first bone 100 with a first fixation plate 102. In one embodiment, said first fixation plate 102 can comprise a portion of a surgical aiming system 111 as illustrated more fully below. In one embodiment, said surgical aiming system 111 can comprise one or more components and methods of using said one or more components. In one embodiment, said surgical aiming system 111 can be used to treat fractures of long bones (such as first bone 100) of said skeletal system 101. In one embodiment, said first bone 100 can comprise a radius bone of said skeletal system 101. Although said skeletal system 101 comprises a human skeletal system, said surgical aiming system 111 can be used on other skeletal systems, such as non-human mammals. For illustrative purposes, said first bone 100 is used in this disclosure, but this use should not be construed as limiting to one among many bones within said skeletal system 101 which may be benefited by the current disclosure. For example, said surgical aiming system 111 can be used on a distal radius 100a, a proximal ulna 100b, a proximal humerus 100c, a distal humerus 100d, a proximal femur 100e, a distal femur 100f, a proximal tibia 100g, or similar as is known in the art.

First bone 100 can comprise a first end 104 (or the proximal radius), a second end 106 (or the distal radius) and a surface 108. First end 104 can comprise a portion of first bone 100 comprising a radial head 112 and a neck 114. Second end 106 can comprise a portion of first bone 100 comprising a syloid process, a baze-carpal articular surface and an ulnar noch, as is known in the art. In one embodiment, said first fixation plate 102 can attach to said surface 108 of said second end 106 of said first bone 100, or in this case the distal radius.

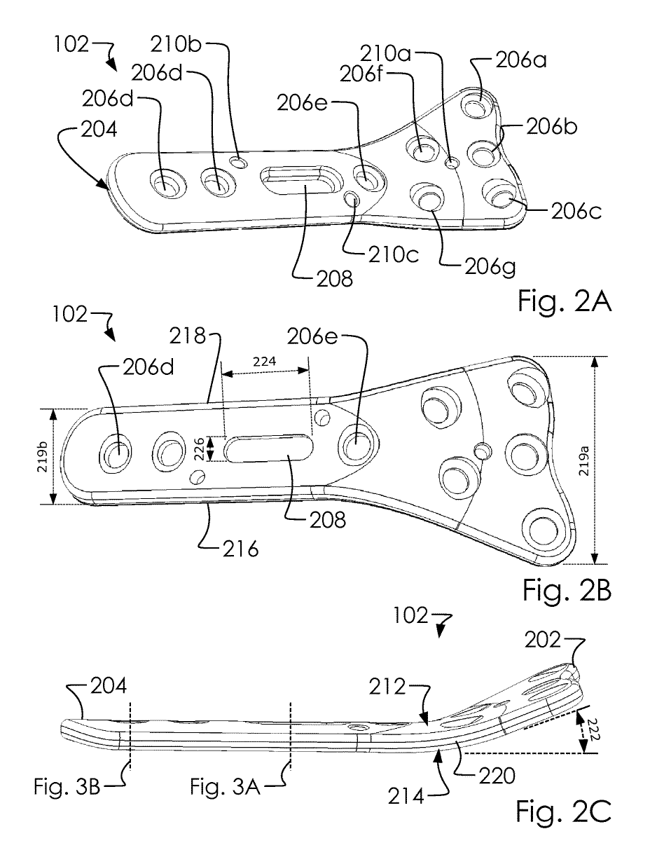

FIGS. 2A, 2B and 2C illustrate a perspective overview, an elevated bottom view and an elevated side view of said first fixation plate 102. In one embodiment, said surgical aiming system 111 can comprise a one or more fixation plates (comprising said first fixation plate 102). Said first fixation plate 102 can comprise a first end 202 and a second end 204. In one embodiment, said first fixation plate 102 can comprise an elongated planar object comprising a substantially smooth surface and rounded edges, adapted to couple with said surface 108 of said first bone 100. In one embodiment, said first end 202 can be broader than said second end 204.

In one embodiment, said first fixation plate 102 can comprise a plurality of plate apertures. In one embodiment, said plurality of plate apertures can each receive a portion of a surgical screw or other binding tool (as discussed infra). In one embodiment, said plurality of plate apertures can comprise a first plate aperture 206a, a second plate aperture 206b, a third plate aperture 206c, a fourth plate aperture 206d, a fifth plate aperture 206e, a sixth plate aperture 206f, a seventh plate aperture 206g, an eighth plate aperture 206h, a central plate aperture 208, a first wire aperture 210a, a second wire 210b, and a third wire aperture 210c.

First fixation plate 102 can comprise a top surface 212, a bottom surface 214, a first side 216 and a second side 218. In one embodiment, said first end 202 can comprise a width 219a and said second end 204 can comprise a width 219b. In one embodiment, said width 219a can be wider than said width 219b. In one embodiment, said first fixation plate 102 can comprise a substantially planar shape with portions of said bottom surface 214 shaped to couple (or mate) with said surface 108 of said first bone 100. Thus, in one embodiment said first fixation plate 102 can comprise a bend 220 between said first end 202 and said second end 204. In one embodiment, as seen from an elevated view of said second side 218, said second end 204 can comprise a substantially straight portion of said first fixation plate 102, followed by said bend 220, and said second side 218 at said first end 202 can be bent up by a bend angle 222, as illustrated.

In one embodiment, some of said plurality of plate apertures (such as first plate aperture 206a, second plate aperture 206b, third plate aperture 206c and fourth plate aperture 206d) can comprise a substantially circular shape. In one embodiment, however, central plate aperture 208 can comprise a substantially elliptical shape comprising a length 224 being longer than a width 226.

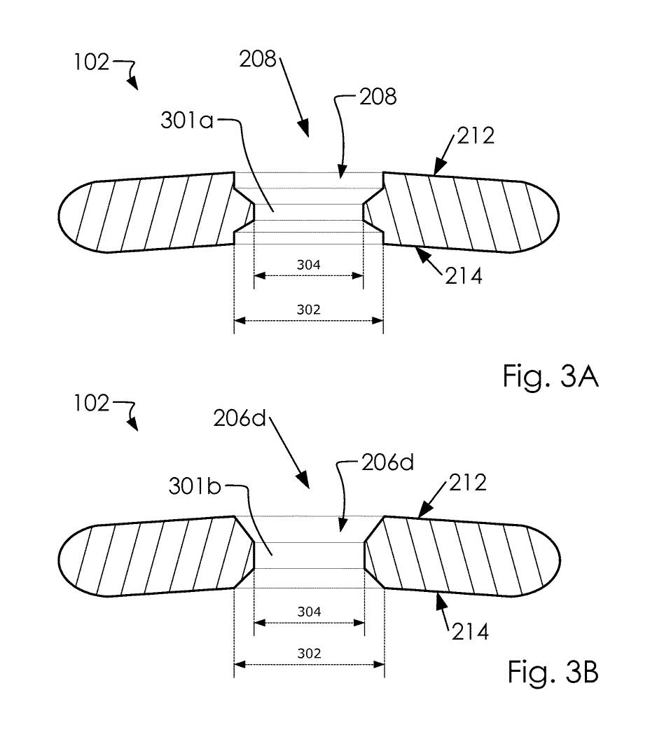

Cross-section cut lines are illustrated in FIG. 2C for FIGS. 3A and 3B, as discussed below.

FIGS. 3A and 3B illustrate an elevated cross-section view of said central plate aperture 208 and said fourth plate aperture 206d, respectively. In one embodiment, said top surface 212 can be substantially convex. In one embodiment, said bottom surface 214 can be substantially concave. In one embodiment, bottom surface 214, being substantially concave, can press against and couple with said surface 108 of said second end 106 of said first bone 100 (being substantially convex). Thus, in one embodiment said first fixation plate 102 can couple with said surface 108 of said first bone 100.

In one embodiment, each among said plurality of plate apertures can comprise an inner lip; e.g., said central plate aperture 208 can comprise a first inner lip 301a and said fourth plate aperture 206d can comprise a second inner lip 301b. In one embodiment, said fourth plate aperture 206d and said central plate aperture 208 can each comprise a first diameter 302. In one embodiment, said first inner lip 301a—being substantially elliptical—can comprise a second diameter 304. However, said central plate aperture 208 and said fourth plate aperture 206d may have different diameters in another embodiment. In one embodiment, said second diameter 304 is wide enough to accommodate a shaft and threading of a screw but narrow enough to catch a head of said screw, as discussed infra, and as is known in the art.

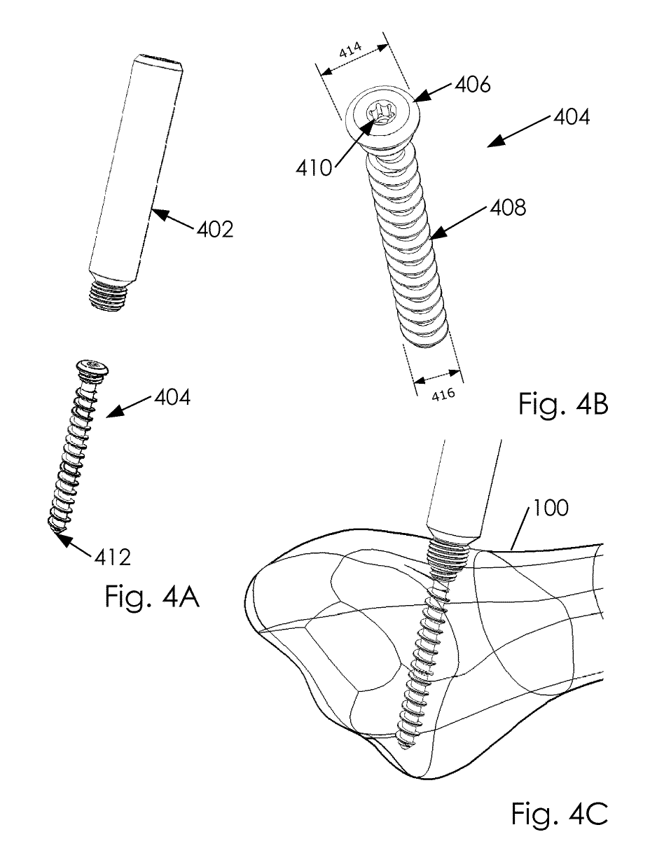

FIGS. 4A, 4B and 4C illustrate a perspective overview of a drill guide 402 with a screw 404, with said drill guide 402, and said screw 404 inserted into a wireframe view of said first bone 100. Said screw 404 can comprise one of a range of well-known surgical screws, pegs, and/or locking or nonlocking implements; wherein, said screw 404 is capable of penetrating and holding a bone (such as first bone 100). In one embodiment, said screw 404 can comprise a head 406 and a threaded portion 408, as is known in the art. In one embodiment, said head 406 can comprise a fitting portion 410. In one embodiment, a portion of said drill guide 402 can attach to said fitting portion 410 and drive said screw 404 in a rotary and linear direction, as is well-known in the art. In one embodiment, said drill guide 402 can be used to guide a drill bit in establishing a pathway prior to affixing a one or more screws. In one embodiment, said screw 404 can comprise a tip 412. In one embodiment, said tip 412 can be sharpened and capable of penetrating first bone 100. In one embodiment, as is known in the art, screwing said screw 404 into said first bone 100 can comprise: aligning said tip 412 on said first bone 100, rotating said screw 404, and pressing said screw 404 into said first bone 100 to a desired depth.

Head 406 can comprise an external diameter 414. Threaded portion 408 can comprise an external diameter 416. In one embodiment, said external diameter 414 is greater than second diameter 304 of said plurality of plate apertures. In one embodiment, said external diameter 416 is smaller than said second diameter 304 of said plurality of plate apertures. Thus, said plurality of plate apertures are capable of allowing a portion of said screw 404 to penetrate said first fixation plate 102 while holding a portion of said first fixation plate 102 against said surface 108 with said head 406 of said screw 404.

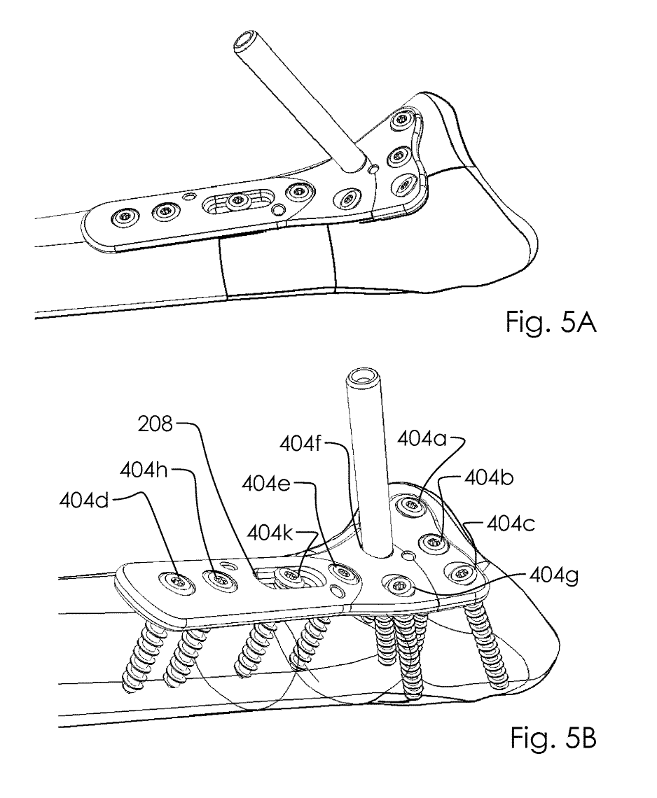

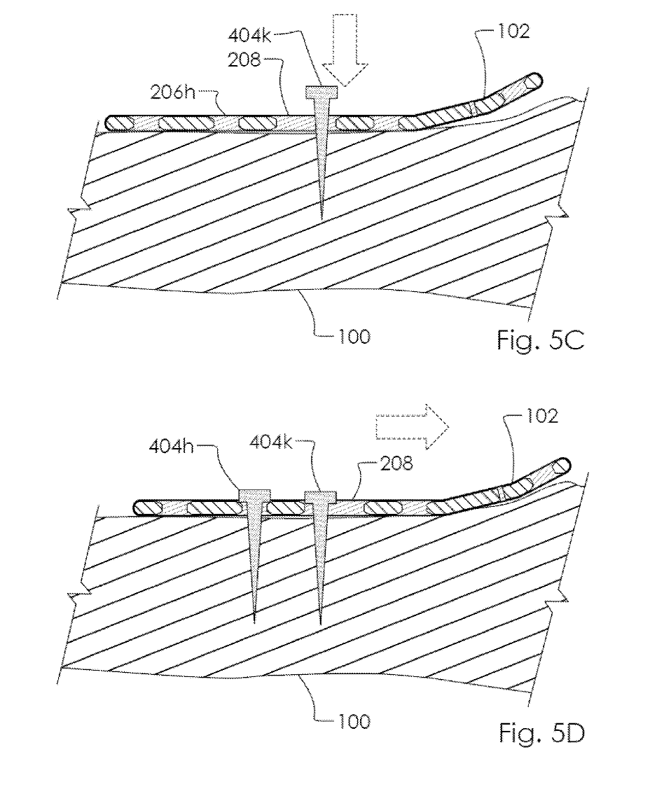

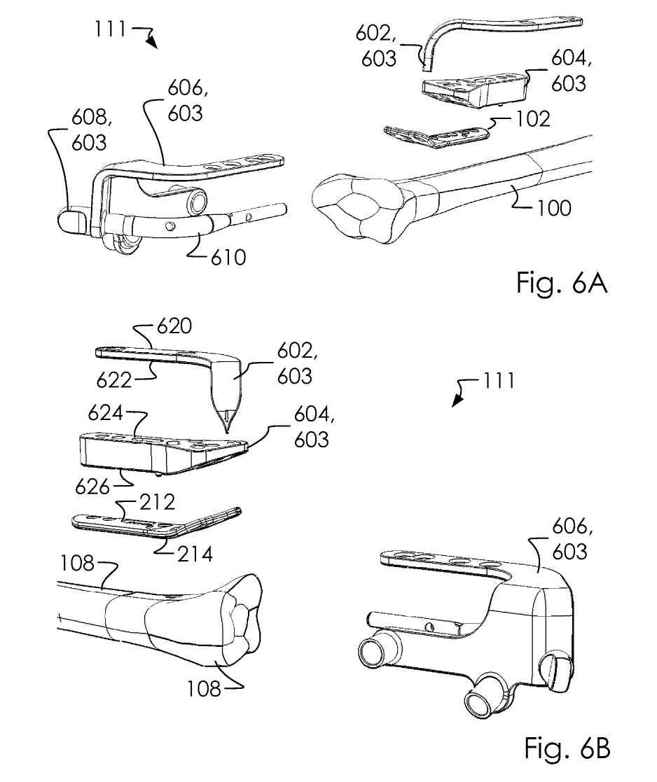

FIGS. 5A and 5B illustrate a perspective overview of said first fixation plate 102 attached to said first bone 100 with a plurality of screws. FIG. 5A illustrates said first bone 100 as a solid view and 5B illustrates said first bone 100 in a wireframe view. In one embodiment, said plurality of screws can comprise a plurality of said screw 404. In one embodiment, attaching said first fixation plate 102 to said first bone 100 can comprise: aligning said first fixation plate 102 with said second end 106 of said first bone 100, and screwing said plurality of screws into said plurality of plate apertures of said first fixation plate 102, this approach represents a prior art method of attaching a plate to a bone. In one embodiment, said bottom surface 214 of said first fixation plate 102 can be substantially flat against said surface 108 of said first bone 100 prior to attaching said first fixation plate 102 to said first bone 100. As stated above, this description represents a standard of care.

Note here that said plurality of screws are inserted into said first bone 100 at a, seemingly, random set of angles relative to one another. However, the positioning, angles and depth of said plurality of screws are the result of careful thought and preparation by surgeons. Consequently, as follows, said surgical aiming system 111 can be used to insert said plurality of screws at predetermined locations and angles relative to other components of said surgical aiming system 111.

In one embodiment, said plurality of screws can comprise a first screw 404a, a second screw 404b, a third screw 404c, a fourth screw 404d, a fifth screw 404e, a sixth screw 404f, a seventh screw 404g, an eighth screw 404h, and said ninth screw 404k.

FIGS. 5C and 5D illustrate a cross-section elevated front view of said first fixation plate 102 and said first bone 100. In one embodiment, screwing said plurality of screws into said plurality of plate apertures can comprise: screwing said ninth screw 404k through said central plate aperture 208 (see FIG. 5C); sliding said first fixation plate 102 toward said second end 106 along said length 224 of said central plate aperture 208 to a final position; tightening said ninth screw 404k; holding said first fixation plate 102 against said first bone 100 with said ninth screw 404k; and screwing a one or more of said plurality of screws into said plurality of plate apertures (such as said eighth screw 404h through said eighth plate aperture 206h, as illustrated in FIG. 5D). Note that said plurality of screws are illustrated in FIGS. 5C and 5D without threading, but this is done for clarity of positioning and should not be construed of a selection of a preferred implement for attaching and holding said first fixation plate 102 to said first bone 100. In a preferred embodiment said plurality of plate apertures have threading, as is known in the art.

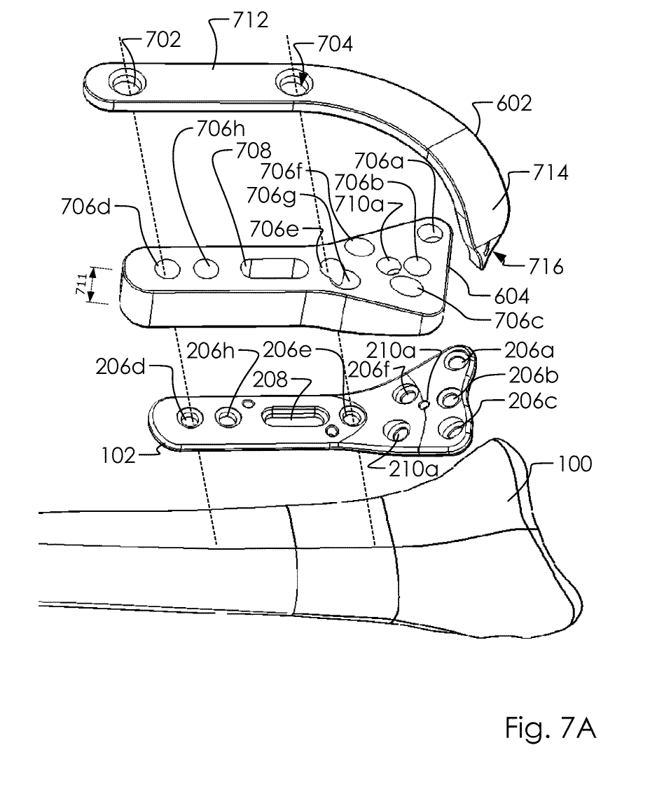

FIGS. 6A and 6B illustrate an exploded perspective first side overview and second side overview of said surgical aiming system 111 with said first bone 100. In one embodiment, said surgical aiming system 111 can comprise of an aiming assembly 603 (comprising of an intramedullary rod guide 602, a channeling assembly 604, a triangulating screw guide 606, and a thumb screw 608), said first fixation plate 102, and a first intramedullary nail 610. In one embodiment, said aiming assembly 603 can comprise of four independent parts or fewer parts, as will be discussed infra).

In one embodiment, said first intramedullary nail 610 can comprise an intramedullary rod (“IM rod”), an intramedullary nail (“IM nail”), an inter-locking nail, or a Küntscher nail. In one embodiment, said first intramedullary nail 610 can comprise a metal rod forced into the medullary cavity of a bone (such as first bone 100). intramedullary nails, like said first intramedullary nail 610, have long been used to treat fractures of long bones of the body, as is well-known in the art.

In one embodiment, each among a one or more components of said surgical aiming system 111 can comprise a plurality of channels which, when held in alignment, are capable of precisely positioning said plurality of screws and said first intramedullary nail 610 when inserted into said first bone 100. Thus, in one embodiment, said one or more components of said surgical aiming system 111 can be used to align and hold said first intramedullary nail 610, as is discussed and illustrated infra.

In one embodiment, said aiming assembly 603 can comprise parts which break apart and are useful for different purposes while a surgeon is attaching said surgical aiming system 111 to said first bone 100. For example, said intramedullary rod guide 602 and said triangulating screw guide 606 may not be used simultaneously, but in sequence, as discussed infra.

Focusing on FIG. 6B, in one embodiment, said intramedullary rod guide 602 can comprise a top surface 620 and a bottom surface 622. Likewise, in one embodiment, said channeling assembly 604 can comprise a top surface 624 and a bottom surface 626. In one embodiment, said bottom surface 622 of said intramedullary rod guide 602 mates with said top surface 624 of said channeling assembly 604; said bottom surface 626 of said channeling assembly 604 mates with said top surface 212 of said first fixation plate 102; and said bottom surface 214 of said first fixation plate 102 mates with said surface 108 of said first bone 100. In one embodiment, the term “mates” means that two components substantially abut one another having a geometry formed to securely stack against one another.

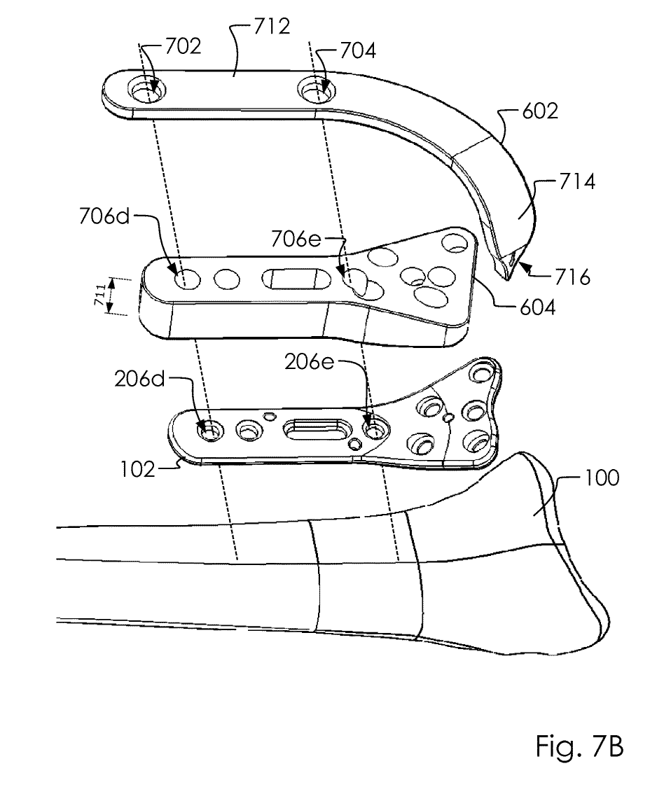

FIGS. 7A, 7B and 7C illustrate two exploded views and an attached perspective overview of said intramedullary rod guide 602, said channeling assembly 604, said first fixation plate 102 and said first bone 100.

In one embodiment, said aiming assembly 603 (here, said channeling assembly 604) can comprise a first channel 706a, a second channel 706b, a third channel 706c, a fourth channel 706d, a fifth channel 706e, a sixth channel 706f, a seventh channel 706g, an eighth channel 706h, a central channel 708, an first wire aperture 710a. Likewise, in one embodiment, said intramedullary rod guide 602 can comprise a first channel 702 and a second channel 704. In one embodiment, channeling assembly 604 can comprise a thickness 711.

In one embodiment, a method of using said surgical aiming system 111 can comprise: placing said first fixation plate 102 on a portion of said first bone 100; stacking said channeling assembly 604 on said first fixation plate 102, and said intramedullary rod guide 602 on top of said channeling assembly 604; aligning a portion of said plurality of channels in said intramedullary rod guide 602 with said channeling assembly 604 and said first fixation plate 102; guiding said plurality of screws through said plurality of channels; and attaching said plurality of screws to said first bone 100 at predetermined positions and angles. In one embodiment, said method of using said surgical aiming system 111 can further comprise: attaching said triangulating screw guide 606 to said channeling assembly 604; inserting said first intramedullary nail 610 into said first bone 100; and attaching said first intramedullary nail 610 on a portion of said triangulating screw guide 606.

In one embodiment, as illustrated in FIG. 7B, aligning and securing said intramedullary rod guide 602, said channeling assembly 604 and said first fixation plate 102 can comprise: aligning said first aperture 702 with said fourth channel 706d and said fourth plate aperture 206d; aligning said second aperture 704 with said fifth channel 706e and said fifth plate aperture 206e; inserting said fourth screw 404d through said first channel 702, said fourth channel 706d and said fourth plate aperture 206d; inserting said fifth screw 404e through said second channel 704, said fifth channel 706e and said fifth plate aperture 206e; and securing said fourth screw 404d and said fifth screw 404e to said first bone 100.

Prior to attaching said intramedullary rod guide 602 and said channeling assembly 604 to said first fixation plate 102, a portion of said first fixation plate 102 can be attached to said first bone 100.

In one embodiment, aligning said surgical aiming system 111 can further comprise: attaching said intramedullary rod guide 602 to said channeling assembly 604; and marking an entry point 718 with a pointing device 716 of said intramedullary rod guide 602; wherein, said entry point 718 can comprise an aligned location for inserting said first intramedullary nail 610. In one embodiment, as illustrated in FIG. 7C, said intramedullary rod guide 602 can be held in alignment with said channeling assembly 604 by inserting one or more of said drill guide 402 through threaded portions of said first aperture 702 and/or said second aperture 704.

Said intramedullary rod guide 602 can comprise an upper portion 712 and a lower portion 714. In one embodiment, said upper portion 712 can comprise a substantially planar and horizontal member and said lower portion 714 can comprise a substantially vertical member of said intramedullary rod guide 602. In one embodiment, viewing said intramedullary rod guide 602 from an elevated side view, said upper portion 712 can be substantially horizontal, then gradually bend downward until lower portion 714 is substantially vertical. In one embodiment, lower portion 714 can comprise said pointing device 716. In one embodiment, said pointing device 716 can comprise marker for said entry point 718 for said first intramedullary nail 610, as discussed infra.

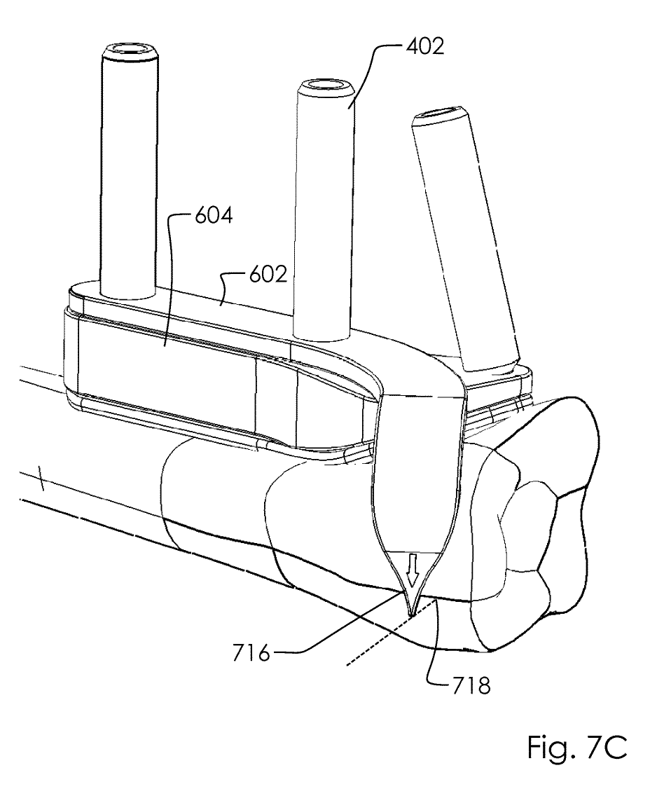

FIGS. 8A and 8B illustrate an exploded perspective front view and overview of said triangulating screw guide 606, said thumb screw 608 and said first intramedullary nail 610. In one embodiment, said thumb screw 608 can comprise a threaded portion 802 and a gripping portion 804. In one embodiment, said triangulating screw guide 606 can comprise a thumb screw socket 806. In one embodiment, said first intramedullary nail 610 can comprise a first end 808 and a second end 810. In one embodiment, said first end 808 can comprise a first coupler 812 and said thumb screw socket 806 can comprise a second coupler 814.

In one embodiment, attaching said first intramedullary nail 610 to said triangulating screw guide 606 can comprise: aligning said threaded portion 802 with said thumb screw socket 806, attaching said first coupler 812 to said second coupler 814, inserting said threaded portion 802 through said thumb screw socket 806 and into said first coupler 812, screwing said threaded portion 802 into said first coupler 812, and tightening said threaded portion 802 of said thumb screw 608 until said triangulating screw guide 606 is firmly held between said thumb screw 608 and said first intramedullary nail 610.

In one embodiment, said triangulating screw guide 606 can comprise a top portion 816 and a side portion 818. In one embodiment, said side portion 818 can comprise a plurality of top apertures. In one embodiment, said plurality of top apertures can comprise a first aperture 820, a second aperture 822 and a third aperture 824.

In one embodiment, said first intramedullary nail 610 can comprise a one or more threaded female apertures each capable of receiving a portion of said threaded portions of said one of said plurality of screws. In one embodiment, said one or more threaded female apertures can comprise a first female aperture 826a, a second female aperture 826b and a third female aperture 826c.

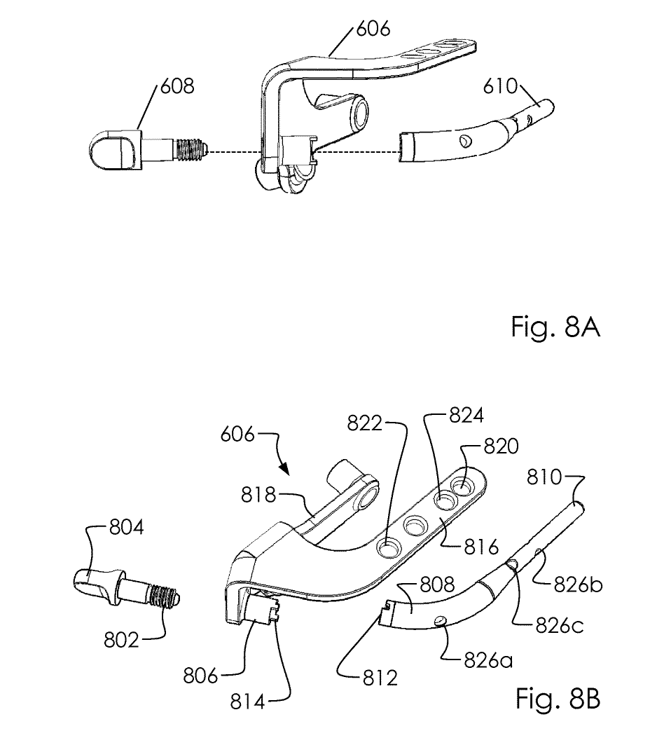

FIG. 9 illustrates a wireframe perspective overview said first intramedullary nail 610 inserted into said first bone 100. In one embodiment, inserting said first intramedullary nail 610 into said first bone 100 can comprise: drilling a portal at said entry point 718, inserting a portion of said first intramedullary nail 610 into a medullary cavity of said first bone 100.

In one embodiment, said plurality of channels of said aiming assembly 603 (here, said channeling assembly 604) are capable of directing said plurality of screws through said first fixation plate 102 and into said first bone 100 at a set of predetermined entry angles. For example, in one embodiment, said second channel 706b can direct said second screw 404b into said first bone 100 at a second entry angle 902b.

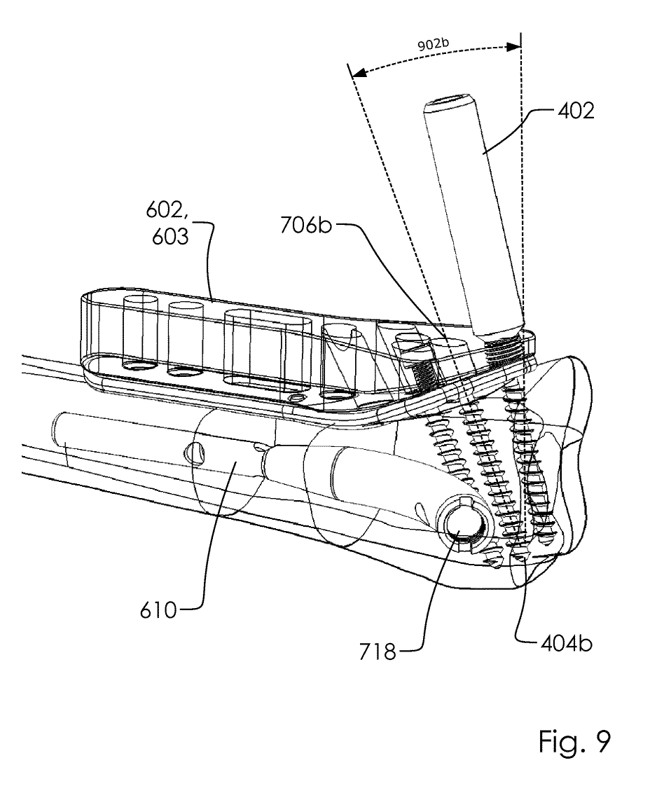

FIGS. 10A and 10B illustrate a perspective lower view and overview of said triangulating screw guide 606, said thumb screw 608 and said first intramedullary nail 610 attached to one another with said first bone 100 and channeling assembly 604 in a wireframe view. In one embodiment, said plurality of said drill guide 402 can comprise a first drill guide 1002 and a second drill guide 1004. In one embodiment, aligning said triangulating screw guide 606 with said channeling assembly 604 can comprise: aligning said first aperture 820 of said triangulating screw guide 606 with said fourth channel 706d, aligning said second aperture 822 with said fifth channel 706e, inserting and attaching said first drill guide 1002 through said first aperture 820 and into fourth channel 706d, and said second drill guide 1004 through said second aperture 822 and into said fifth channel 706e.

In one embodiment, attaching said triangulating screw guide 606 to said channeling assembly 604 and said first intramedullary nail 610 can comprise: aligning said triangulating screw guide 606 with said channeling assembly 604, attaching said triangulating screw guide 606 to said channeling assembly 604 with said first drill guide 1002 and said second drill guide 1004, insert said thumb screw 608 through said thumb screw socket 806, attaching said thumb screw 608 to said first intramedullary nail 610, and holding said triangulating screw guide 606 between said thumb screw 608 and said first intramedullary nail 610.

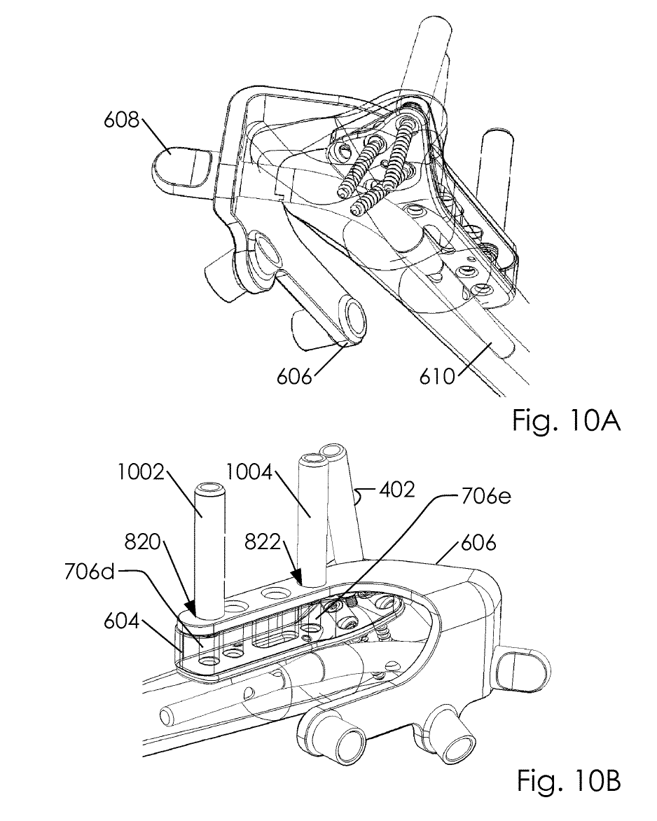

FIGS. 11A and 11B illustrate a perspective overview and a perspective lower view of said triangulating screw guide 606. In one embodiment, said triangulating screw guide 606 can comprise a plurality of channels capable of aligning a portion of said plurality of screws and/or drilling tools with said one or more threaded female apertures of said first intramedullary nail 610.

In one embodiment, said plurality of channels of said triangulating screw guide 606 can comprise a first channel 1102a (having a first axis 1104a), a second channel 1102b (having a second axis 1104b) and said third aperture 824 (having a third axis 1104c).

Said side portion 818 of said triangulating screw guide 606 can comprise one or more side apertures. In one embodiment, said one or more side apertures can comprise a first aperture 1108a (comprising said first channel 1102a) and a second aperture 1108b (comprising said second channel 1102b).

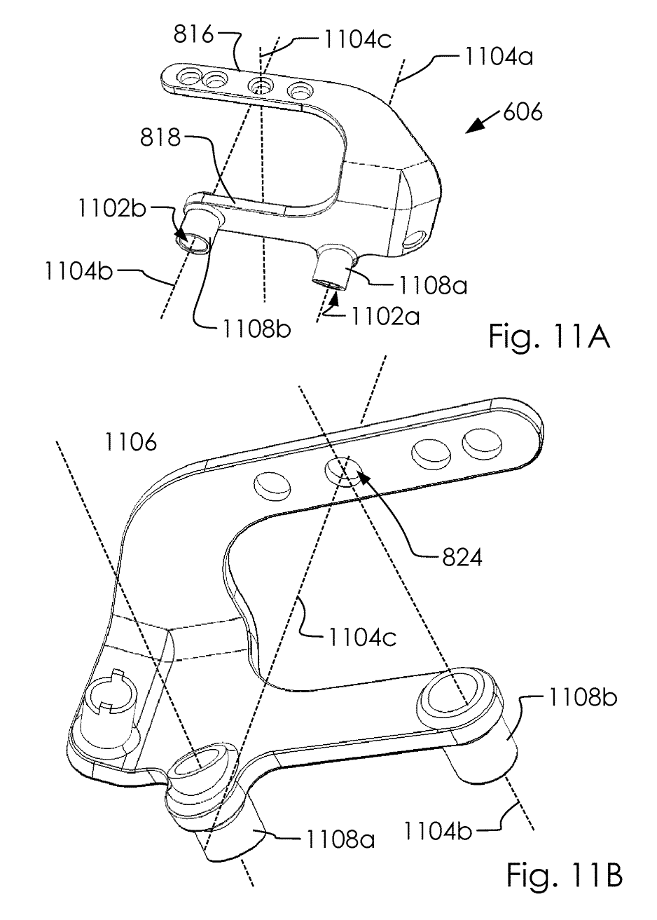

FIGS. 12A and 12B illustrate a perspective side view and a perspective front view of said first intramedullary nail 610 attached to said triangulating screw guide 606 with three of said plurality of screws. In one embodiment, said plurality of screws can comprise an eleventh screw 1202, a twelfth screw 1204 and a thirteenth screw 1206. In one embodiment, said first axis 1104a aligns with said first female aperture 826a, said second axis 1104b aligns with said second female aperture 826b, and said third central axis 1104c aligns with said third female aperture 826c. In one embodiment, attaching said thirteenth screw 1206 can comprise the same screw as said ninth screw 404k.

In one embodiment, a method of using said surgical aiming system 111 can comprise: inserting said first intramedullary nail 610 into said first bone 100; attaching said triangulating screw guide 606 to said first intramedullary nail 610; aligning and inserting a portion of said plurality of screws with/through said plurality of channels of said triangulating screw guide 606; locating said one or more threaded female apertures with said portion of said plurality of screws, and securing said first fixation plate 102 and said first intramedullary nail 610 to one another in a relatively fixed position. The benefit of this feature is substantial, as said first intramedullary nail 610 is inside of said first bone 100, and said first bone 100 is within an arm of a patient; thus, using said plurality of channels to find said one or more threaded female apertures on said first intramedullary nail 610 can comprise a useful tool to surgeons.

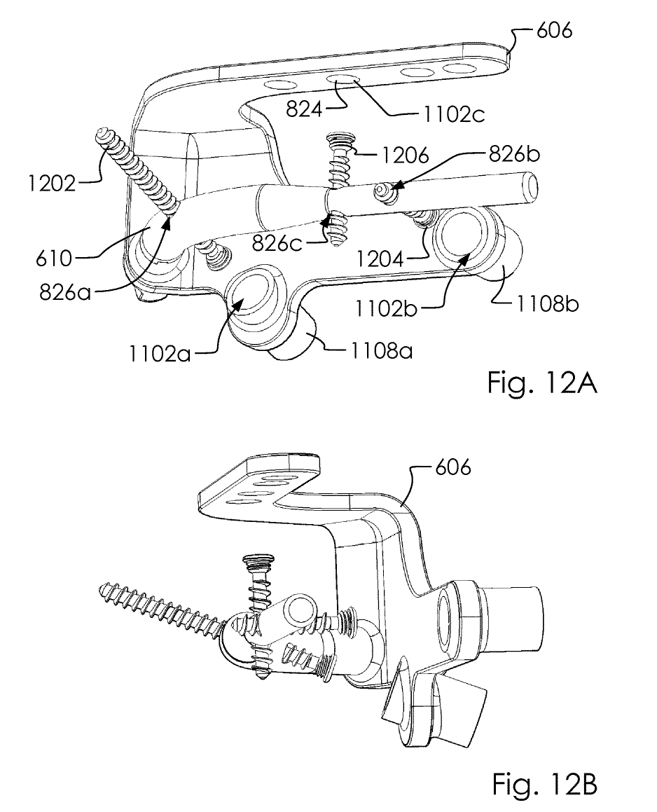

FIGS. 13A, 13B, 13C, 13D and 13E illustrate a series of perspective overviews of a stacked drilling system 1300. In one embodiment, said stacked drilling system 1300 can comprise a tissue sleeve 1302, a drill sleeve 1304, and a trocar 1306. In one embodiment, said surgical aiming system 111 can comprise a one or more of said stacked drilling system 1300. In one embodiment, each of said stacked drilling system 1300 can slide through said first aperture 1108a and said second aperture 1108b. In one embodiment, attaching plurality of screws through said first aperture 1108a and/or said second aperture 1108b can comprise preparing a plurality of apertures in said first bone 100 for said plurality of screws. In one embodiment, preparing a plurality of apertures in said first bone 100 for said plurality of screws can comprise: inserting said tissue sleeve 1302 into said first aperture 1108a or said second aperture 1108b; pressing and/or drilling said tissue sleeve 1302 through a tissue of said patient's arm; inserting a portion of said drill sleeve 1304 through said tissue sleeve 1302; pressing and/or drilling said drill sleeve 1304 into said arm; inserting a portion of said trocar 1306 through said drill sleeve 1304; pressing and/or drilling said trocar 1306 down to said first bone 100; removing said trocar 1306; drilling through said drill sleeve 1304; removing said drill sleeve 1304 from said tissue sleeve 1302; and screwing said eleventh screw 1202 and/or thirteenth screw 1206 through said tissue sleeve 1302 and into said one or more threaded female apertures of said first intramedullary nail 610.

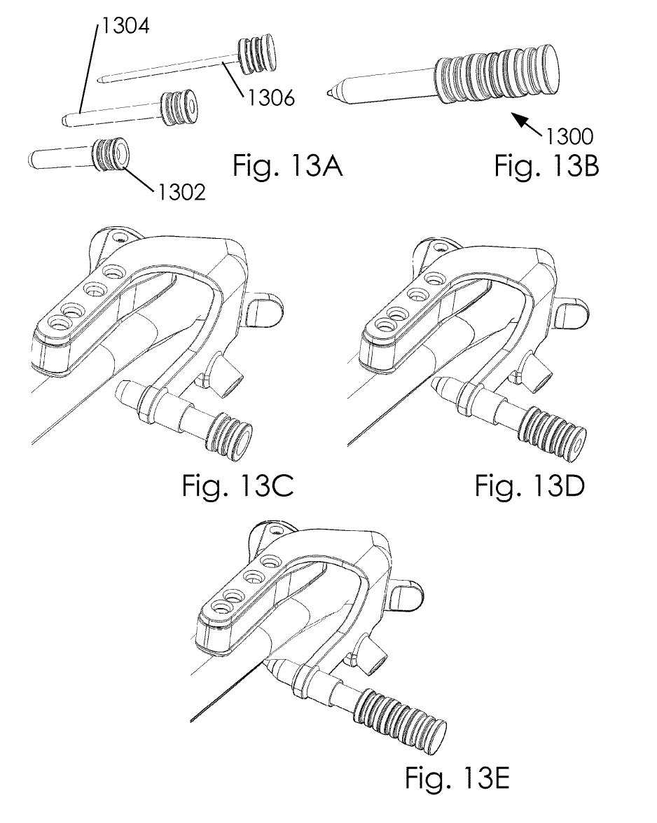

FIGS. 14A and 14B illustrate a wireframe view and a solid view of a perspective overview of said first bone 100 comprising said first intramedullary nail 610 inserted and attached to said first fixation plate 102 with said channeling assembly 604, said triangulating screw guide 606, and said thumb screw 608.

In one embodiment, said thirteenth screw 1206 can be configured to affix said an extramedullary location (said first fixation plate 102) with an intramedullary location (said third female aperture 826c of said first intramedullary nail 610) by using said channeling assembly 604 and either said triangulating screw guide 606 or said intramedullary rod guide 602.

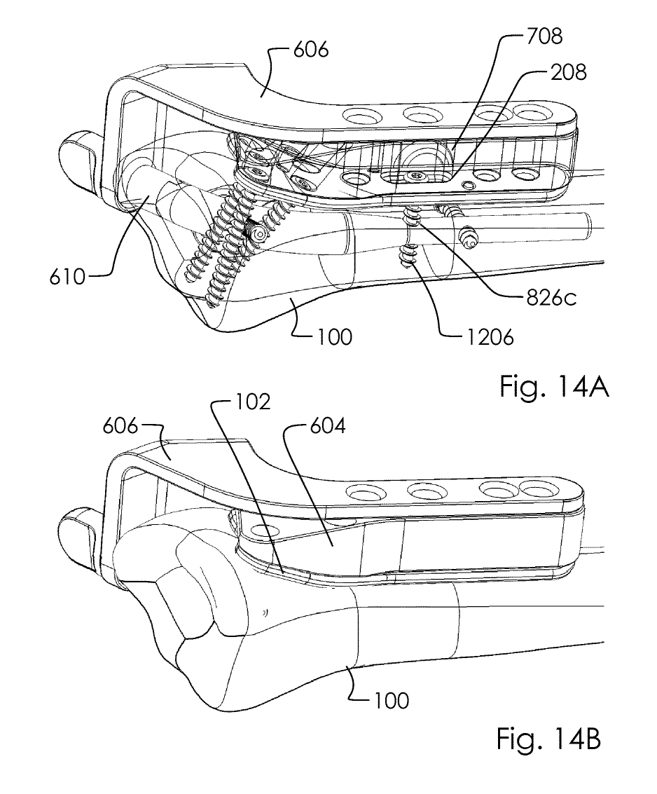

FIG. 15 illustrates a perspective overview of said first bone 100 with said first fixation plate 102 attached, said first intramedullary nail 610 inserted, and a portion of said plurality of screws installed. In one embodiment, said surgical aiming system 111 can comprise: installing said first fixation plate 102, said first intramedullary nail 610 and said plurality of screws to holding said surgical aiming system 111 to said first bone 100.

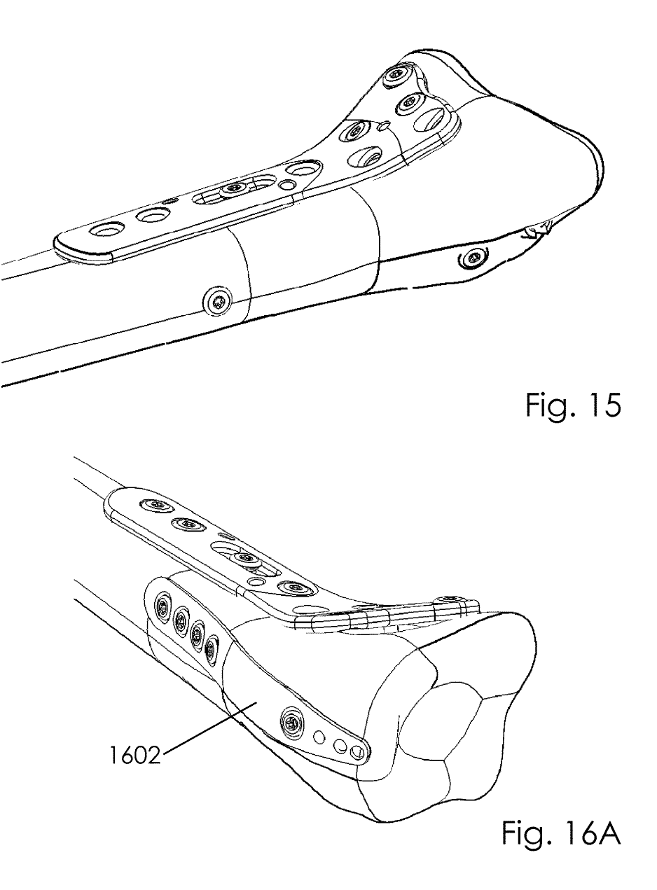

FIGS. 16A, 16B and 16C illustrate a perspective overview said surgical aiming system 111 attached to said first bone 100 with said first fixation plate 102 and a second fixation plate 1602.

In one embodiment, said surgical aiming system 111 can comprise a one or more plates attached to said first bone 100. In one embodiment, said one or more plates can hold a portion of said first bone from a plurality of surface locations around one or more nails within said first bone. In one embodiment, said one or more plates attached to said first bone 100 can comprise said first fixation plate 102 and a second fixation plate 1602. In one embodiment, said second fixation plate 1602 can support a styloid portion of said first bone 100 while said first fixation plate 102 can support a top portion of said first bone 100. In one embodiment, said second fixation plate 1602 and said first fixation plate 102 can attach around said first bone 100 as illustrated (substantially at a perpendicular angle relative to said first intramedullary nail 610) or at differing angles relative to said first intramedullary nail 610. In one embodiment, said second fixation plate 1602 can be useful for reconstructing said first bone 100, where additional support is required in an operation. In one embodiment, said second fixation plate 1602 can comprise one or more apertures capable of receiving one or more of said plurality of screws, as illustrated.

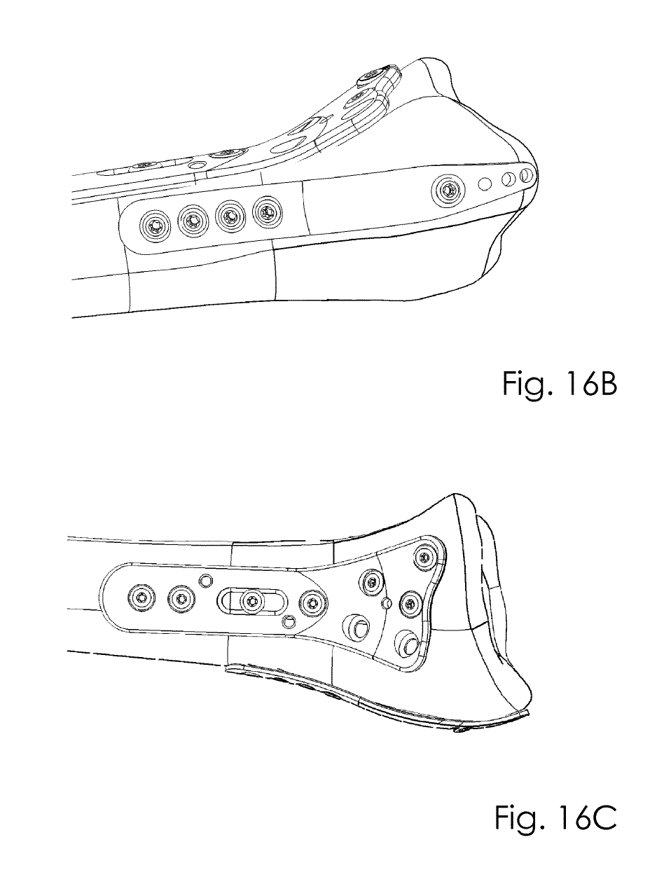

FIGS. 17A, 17B, 17C and 17D illustrate an elevated side view, an elevated top view, an elevated front view and a perspective lower view of a triangulating screw guide 1700. In one embodiment, said triangulating screw guide 1700 can be substantially identical to said triangulating screw guide 606, but-for a rearrangement of said channels, as illustrated. In one embodiment, a multi-channel aperture 1702 can provide for the direction of a one or more screws into said second fixation plate 1602, as illustrated infra.

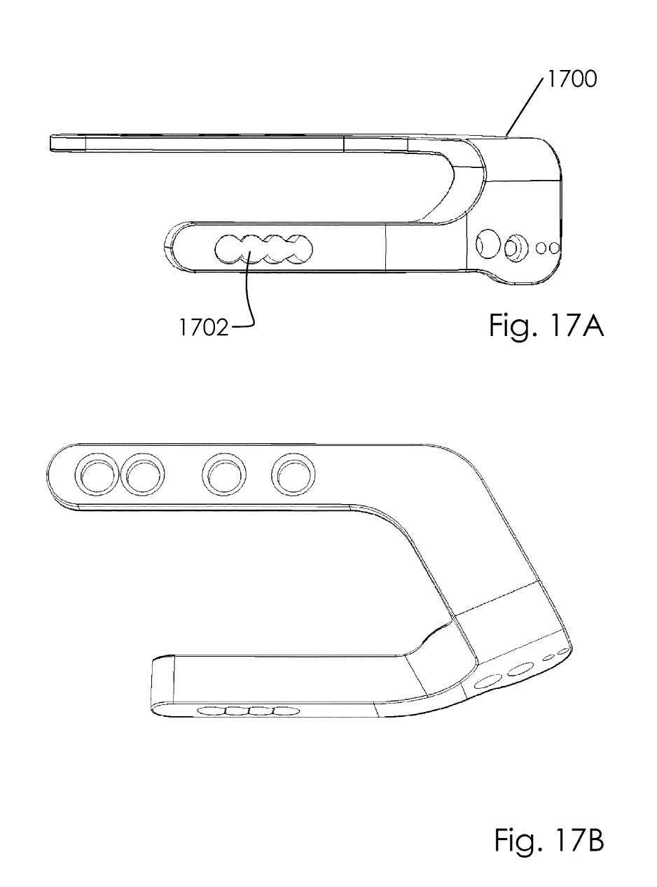

FIGS. 18A and 18B illustrate a series of perspective overviews of said triangulating screw guide 1700 aligned with said first fixation plate 102 and said second fixation plate 1602. In one embodiment, said second embodiment of said surgical aiming system 111 can comprise said first fixation plate 102, said second fixation plate 1602, said channeling assembly 604, said triangulating screw guide 1700, and said plurality of screws. In one embodiment, attaching said second embodiment of said surgical aiming system 111 can comprise: attaching said first fixation plate 102 to said first bone 100; aligning said second fixation plate 1602 with said styloid portion of said first bone 100; aligning said channeling assembly 604 and said triangulating screw guide 1700 with said first fixation plate 102; attaching said second fixation plate 1602 to said first fixation plate 102.

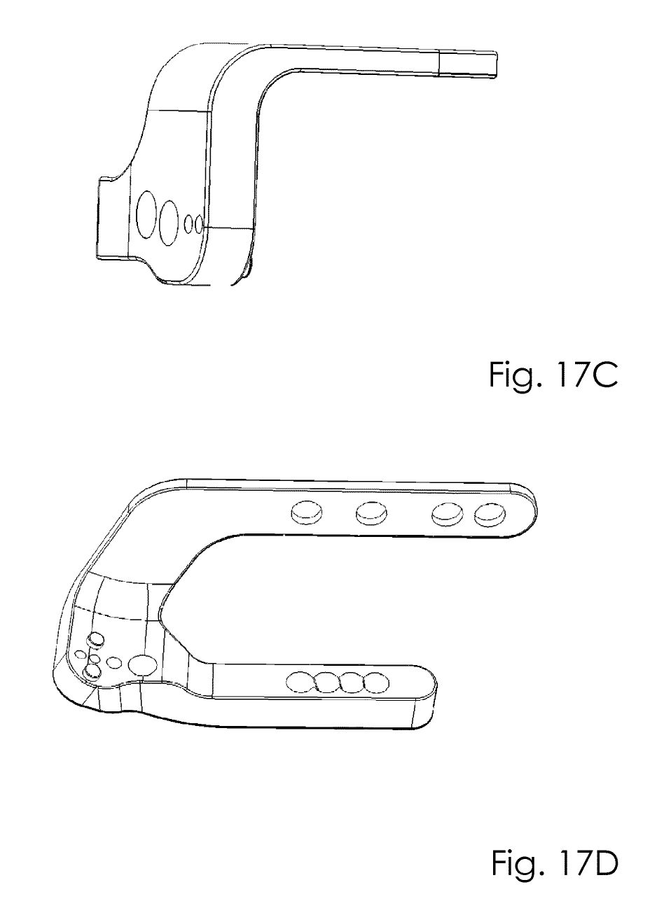

FIGS. 19A and 19B illustrate a perspective wireframe overview and detailed view of said triangulating screw guide 1700, second fixation plate 1602, a one or more nails, and said first fixation plate 102 attached to said first bone 100. In one embodiment, said triangulating screw guide 1700 can be used to insert said one or more nails (comprising a first intramedullary nail 1902a, a second intramedullary nail 1902b and a third intramedullary nail 1902c) into said first bone 100.

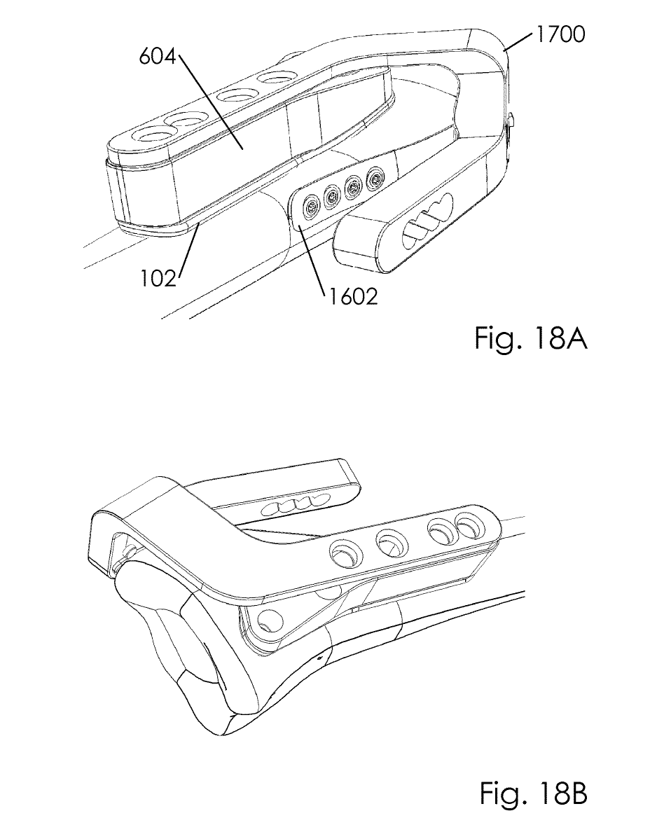

FIGS. 20A and 20B illustrate a perspective overview of a first aiming assembly 2000, said first fixation plate 102 and a first bone 100. In one embodiment, said first aiming assembly 2000 can comprise an integrated component comprising parts previously introduced as said channeling assembly 604 and said intramedullary rod guide 602. In one embodiment, said first aiming assembly 2000 can comprise said aiming assembly 603 in one fully integrated part. Accordingly, said surgical aiming system 111 can comprise: a one or more fixation plates (such as said first fixation plate 102) capable of attaching extramedullary to said first bone 100, said aiming assembly 603 (such as said first aiming assembly 2000) capable of attaching intramedullary to sad first bone 100, and a plurality of screws to attach them to one another; wherein, said surgical aiming system 111 is capable of holding said extramedullary and intramedullary components in a relatively fixed position to one another during assembly of said surgical aiming system 111.

In one embodiment, said surgical aiming system (such as said first aiming assembly 2000) can comprise a one-piece component.

In one embodiment, said first aiming assembly 2000 can comprise a spacing portion 2002, a pointing device 2004, and a plurality of channels (such as said first channel 706a and said central channel 708). In one embodiment, said first aiming assembly 2000 can be used in conjunction with a one or more fixation plates (such as said first fixation plate 102).

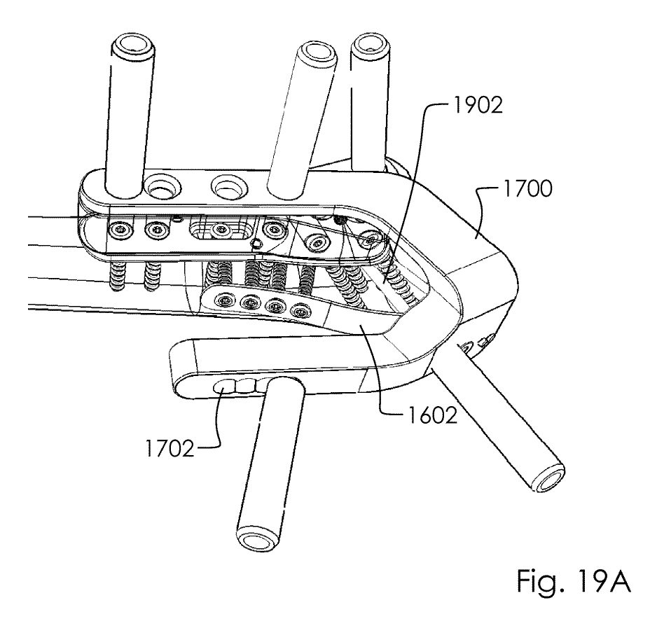

FIGS. 21A and 21B illustrate a perspective overview of a second aiming assembly 2100, said first bone 100 and said first fixation plate 102. In one embodiment, said second aiming assembly 2100 can be similar to said first aiming assembly 2000, in that it is an integral component used in lieu of a plurality of components as illustrated with said channeling assembly 604 and said triangulating screw guide 606. In one embodiment, said second aiming assembly 2100 can comprise one of said aiming assembly 603, as discussed for said first aiming assembly 2000, supra. In one embodiment, said second aiming assembly 2100 can comprise a spacing portion 2102 and a triangulating screw guide portion 2104.

In one embodiment, holding said extramedullary and intramedullary components said surgical aiming system 111 in a relatively fixed position to one another can comprise: attaching said one or more fixation plates (such as said first fixation plate 102) extramedullary to said first bone 100; attaching said aiming assembly 603 (such as said second aiming assembly 2100) intramedullary to sad first bone 100 via a plurality of screws.

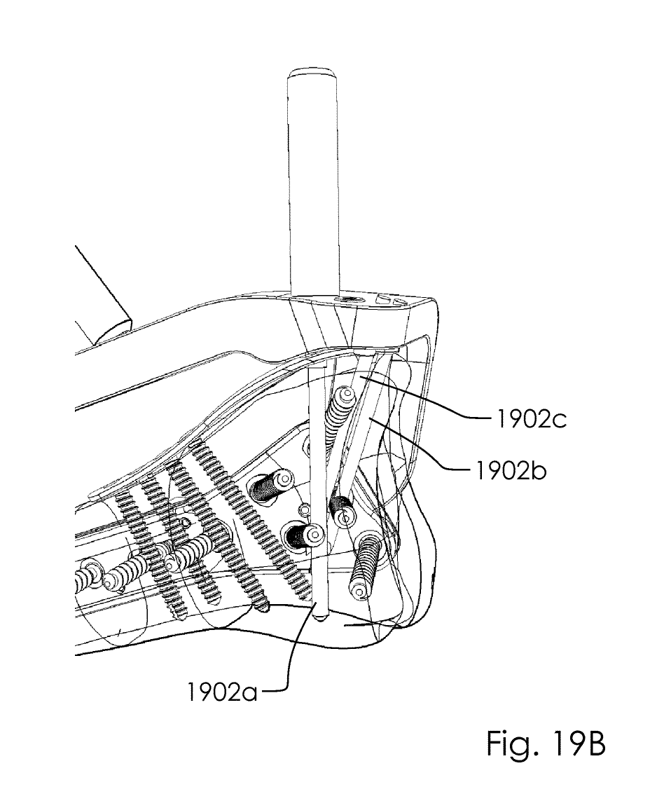

FIGS. 22A and 22B illustrate a perspective overview of a channeling assembly 2200, said first bone 100 and said first fixation plate 102. In one embodiment, said channeling assembly 2200 can be similar to said channeling assembly 604 in that excess material between a one or more channels is absent. In one embodiment, said channeling assembly 2200 can be used in lieu of said channeling assembly 604 while saving on materials cost and weight.

In one embodiment, said channeling assembly 2200 can comprise a frame 2202 and a plurality of channels 2204 (such as a first channel 2204a). In one embodiment, said plurality of channels are capable of mating with said first fixation plate 102. For example, in one embodiment, said first channel 2204a (having a lower portion 2206a) can align with a first aperture 2208a in said first fixation plate 102. In one embodiment, said channeling assembly 2200 can be used with a one or more fixation plates or a one or more aiming assemblies.

Likewise, in one embodiment, said pointing device 2004 and/or said triangulating screw guide portion 2104 can be attached to said channeling assembly 2200. Thus, said channeling assembly 2200 is included as an exemplary embodiment for said channeling assembly 604 and illustrates that a solid block need not be employed to provide the benefits of said channeling assembly 604 and the overall benefits of said surgical aiming system 111.

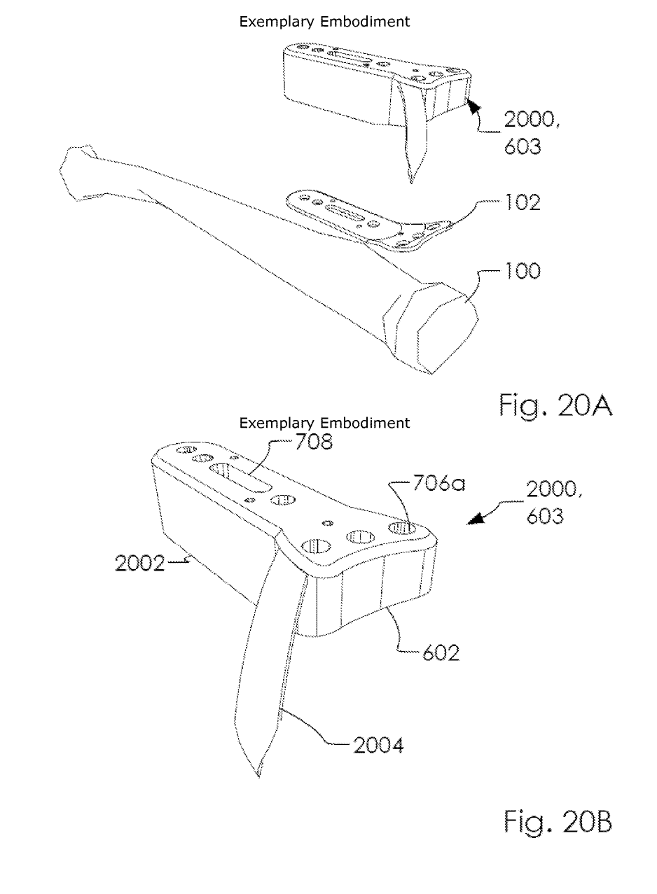

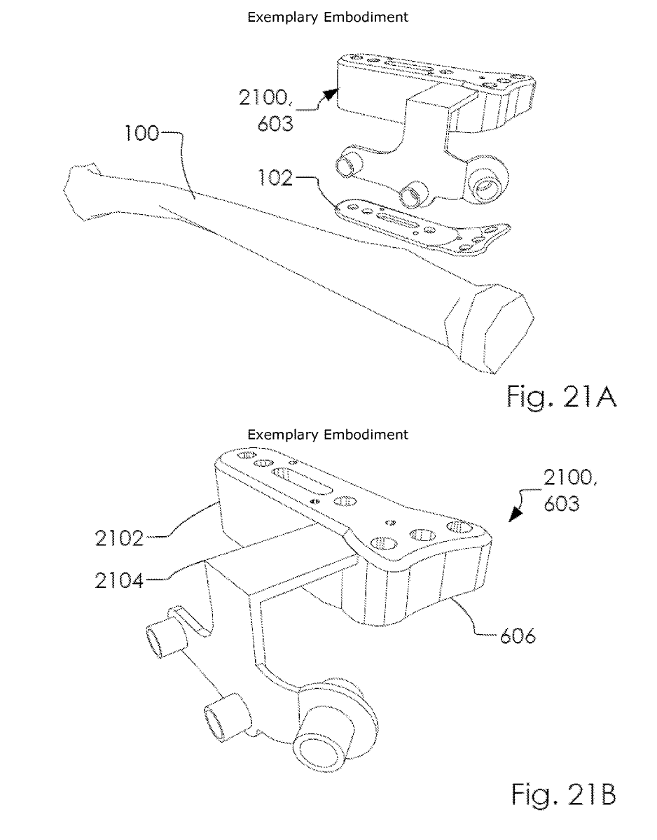

FIGS. 23A, 23B and 23C illustrate a perspective side view of said surgical aiming system 111. FIG. 23A includes said first bone 100 and FIGS. 23B-23C do not include said first bone 100.

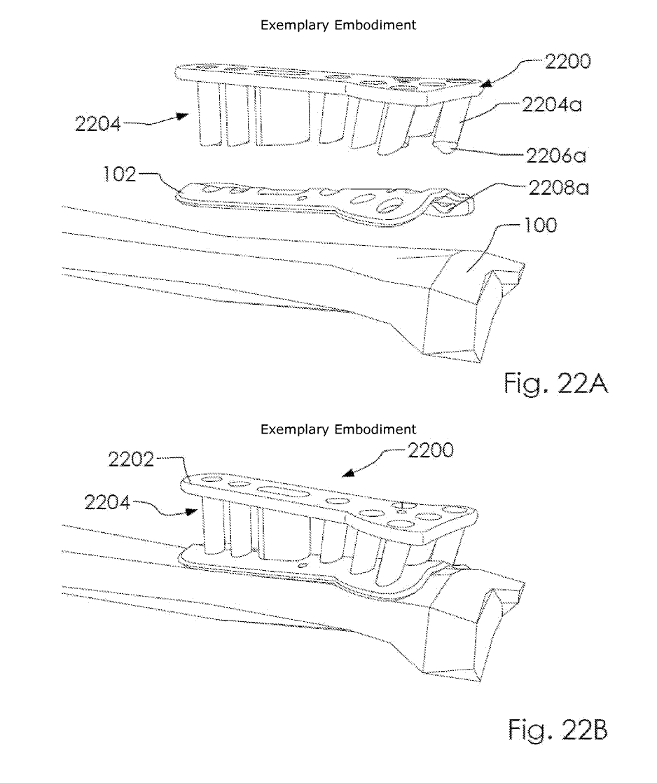

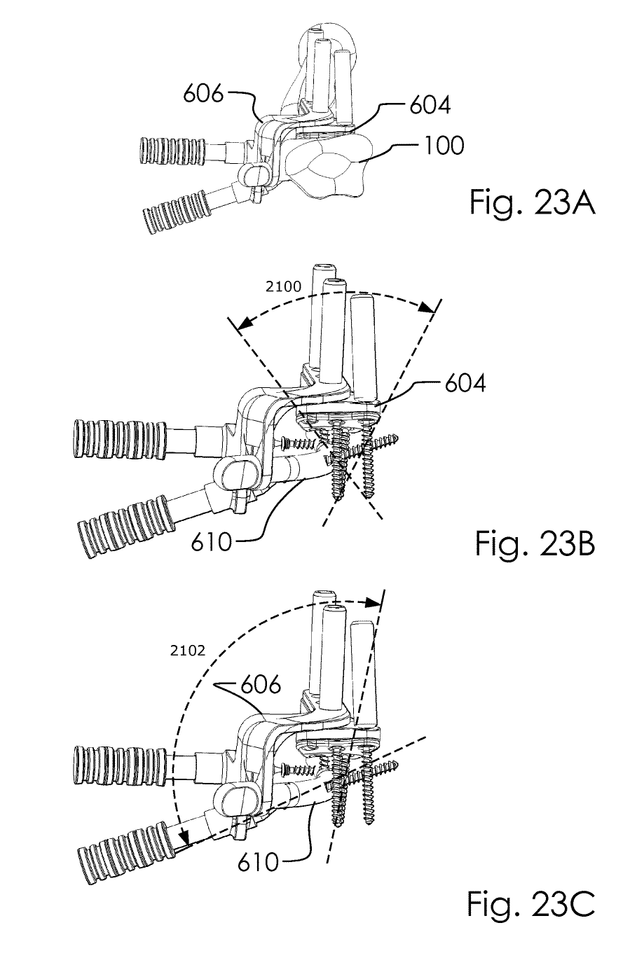

In one embodiment, said surgical aiming system 111 can be used to triangulate locations within said first bone 100, as discussed supra and illustrated here. For example, in one embodiment, said channeling assembly 604 and said triangulating screw guide 606 can be used to place said plurality of screws and/or said first intramedullary nail 610 (or similar) within said first bone 100 in a precise location. Said surgical aiming system 111 can be adapted to triangulate a channel through portions of said triangulating screw guide 606 and/or said channeling assembly 604 to place said screws and/or nails as illustrated in FIGS. 24A and 24B (more infra). However, in FIG. 23A-23C, it is established that said channeling assembly 604 can triangulate into said first bone 100 throughout a first radial range 2100. In one embodiment, said first radial range 2100 can correspond to a range of angles corresponding to said channeling assembly 604. Likewise, in one embodiment, said triangulating screw guide 606 can provide a second radial range 2102 into said first bone 100. In one embodiment, said second radial range 2102 can wrap substantially around said first bone 100 and therefore provide for complex angles of entry and locations. As illustrated here, said triangulating screw guide 606 wraps substantially 120 degrees around said first bone 100 and said first intramedullary nail 610, but it is disclosed and claimed that said triangulating screw guide 606 can be constructed to wrap substantially around said first bone 100. Accordingly, in one embodiment, said surgical aiming system 111 can be used to triangulate and link intramedullary and extramedullary locations and angles so as to provide for accurate and precise care to patients.

FIGS. 24A and 24B illustrate a perspective lower view of said surgical aiming system 111 without said first bone 100. FIG. 24A includes said plurality of screws and FIG. 24B does not.

It has been claimed and described that said surgical aiming system 111 allows a user to triangulate locations as between intramedullary and extramedullary locations. In one embodiment, such locations can be linked as described herein. As illustrated, said second female aperture 826b of said first intramedullary nail 610 is an intramedullary location, and said second channel 1102b and said is in an extramedullary location, even though said first bone 100 is not shown in these figures. In one embodiment, said second channel 1102b can comprise a first vertex 2202 and a second vertex 2204, each corresponding to the end points of said second channel 1102b. In one embodiment, a portion of said first intramedullary nail 610 (such as said gripping portion 804) is in an extramedullary location, and that portion can comprise a third vertex 2206. In one embodiment, said second female aperture 826b can represent a fourth vertex 2208. Since the dimensions and distances between said first vertex 2202, said third vertex 2206 and said fourth vertex 2208 are known, these locations being intramedullary and extramedullary can be known. Since said first vertex 2202 and said second vertex 2204 can be designed to be in line with said fourth vertex 2208, placing said eleventh screw 1202 through said second channel 1102b and into said first bone 100 will ensure that said eleventh screw 1202 will attach to said second female aperture 826b.

Likewise, and as discussed in the description of FIGS. 14A and 14B, said thirteenth screw 1206 can be used to create a link between said first fixation plate 102 and all of said triangulating screw guide 606, said first intramedullary nail 610 and said and those parts generally referred to as said aiming assembly 603.

In one embodiment, the process of triangulating said plurality of screws between said extramedullary location and said intramedullary location can comprise the use of the parts of said surgical aiming system 111 for this purpose. Granted, it could be construed that said head 406 of said plurality of screws are the extramedullary location and the nail aperture (such as said third female aperture 826c) are the intramedullary location and said channeling assembly 604 links the two locations. To this end, in one embodiment, said thirteenth screw 1206 can be configured to affix said an extramedullary location (said first fixation plate 102) with an intramedullary location (said third female aperture 826c of said first intramedullary nail 610) by using said channeling assembly 604 and either said triangulating screw guide 606 or said intramedullary rod guide 602.

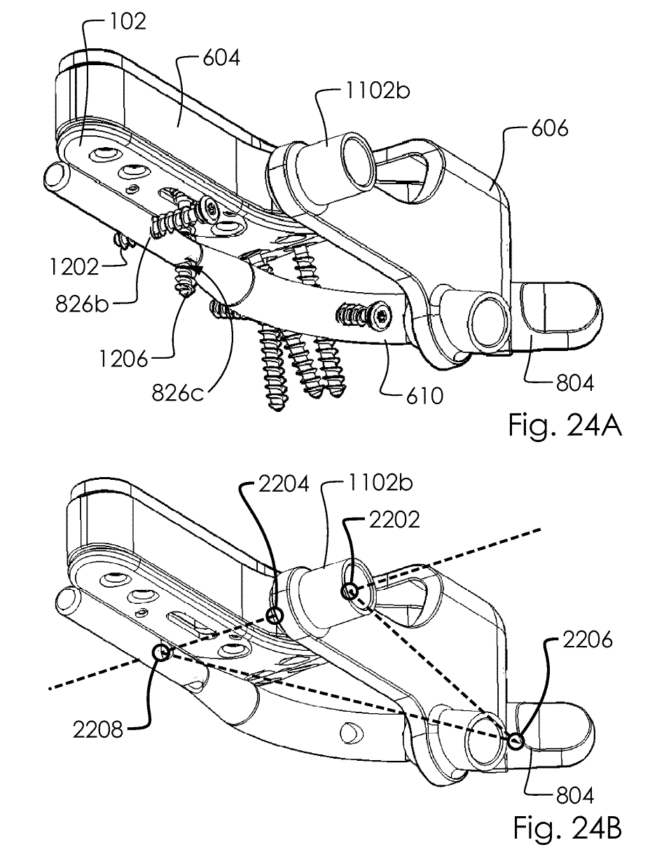

FIGS. 25A and 25B illustrate perspective lower view and overview of said triangulating screw guide 606, said thumb screw 608 and an alternative nail 2500 attached to one another with said first bone 100 and channeling assembly 604 in a wireframe view.

In one embodiment, said alternative nail 2500 can be substantially identical to said first intramedullary nail 610 but for the addition of a first aperture 2502 and a second aperture 2504. Said first intramedullary nail 610 and said alternative nail 2500 can be referred to collectively as an “intramedullary nail”.

In one embodiment, said first aperture 2502 and said second aperture 2504 can each be configured to accept one among said plurality of screws (such as said screw 404). For example, in one embodiment, said first aperture 820 can accept and align said first drill guide 1002 with said first aperture 2502; and said second aperture 822 can accept and align said second drill guide 1004 with said second aperture 2504. In one embodiment, said channeling assembly 604 can include said plurality of channels for placing said plurality of screws through said first fixation plate 102. In this embodiment, said fourth channel 706d can be configured to be aligned with said first drill guide 1002; and therefore, said fourth plate aperture 206d in said first fixation plate 102 can be aligned with said first aperture 2502 in said alternative nail 2500. In one embodiment, said fifth channel 706e can be configured to align and with said second drill guide 1004 when said fourth channel 706d is aligned with said first drill guide 1002. Thus, in one embodiment said fifth plate aperture 206e can be aligned with said second aperture 2504, and said screw 404 can be inserted through both of said first fixation plate 102 and said alternative nail 2500; further, securing said first fixation plate 102 and said alternative nail 2500 to one another.

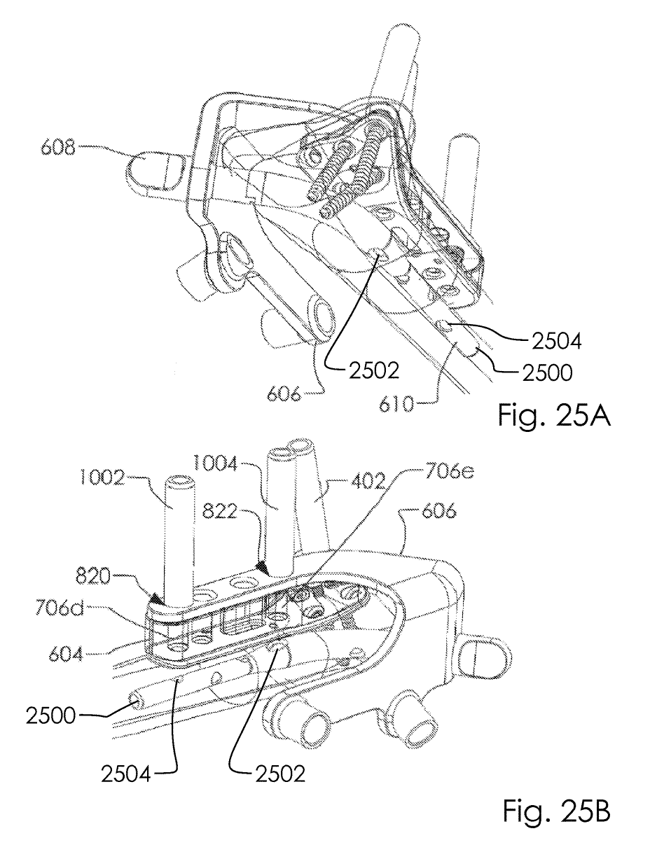

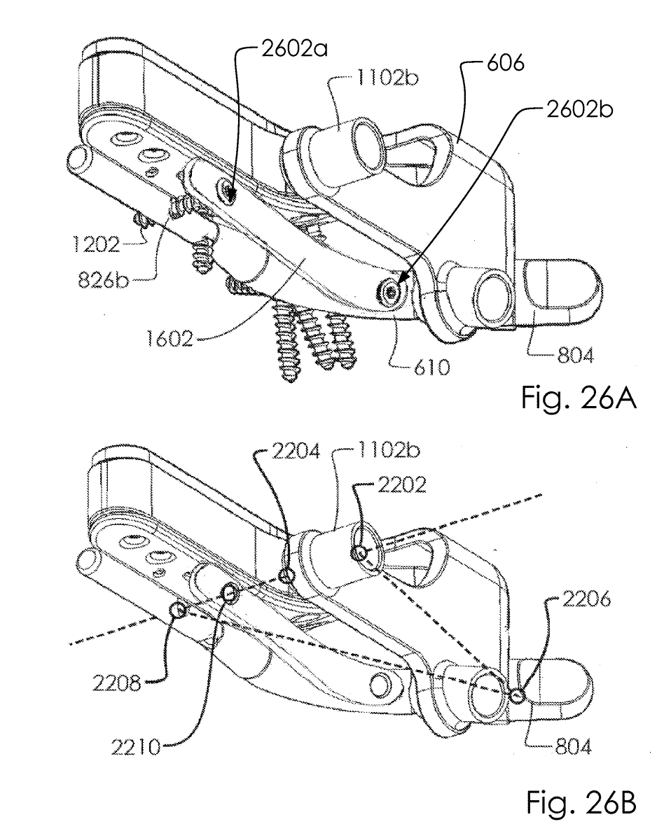

FIGS. 26A and 26B illustrate a perspective lower view of said surgical aiming system 111 without said first bone 100. FIG. 26A includes said plurality of screws and FIG. 26B does not.

Note that FIGS. 26A-26B are similar to FIGS. 24A-24B, but-for the additional element of said second fixation plate 1602. In one embodiment, said second fixation plate 1602 and said first fixation plate 102 can comprise portions of said one or more plates.

In one embodiment, said surgical aiming system 111 can align and triangulate said plurality of screws as through said one or more plates and said first intramedullary nail 610. For example, in one embodiment, said second fixation plate 1602 can comprise a first aperture 2602a and a second aperture 2602b. In one embodiment, said first aperture 2602a can comprise a fifth vertex 2210. In one embodiment, said fifth vertex 2210 can be in line with said first vertex 2202, said second vertex 2204 and said fourth vertex 2208. Accordingly, surgical aiming system 111 can be configured to align one or more plates with said first intramedullary nail 610 by triangulation. Note here that said second fixation plate 1602 can be attached to said first bone 100 using drill guides as discussed supra.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other or in another anatomic location with appropriately configured hardware and guides. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. A surgical aiming system comprising:
a one or more fixation plates, an aiming assembly, a one or more nails and a plurality of screws;
said one or more fixation plates comprising a first fixation plate;
said one or more nails comprising a first intramedullary nail;
said first intramedullary nail comprising a one or more apertures comprising a first aperture;
said aiming assembly comprising a channeling assembly having a plurality of channels;
said one or more fixation plates comprising a plurality of plate apertures configured to receive a portion of said plurality of screws;
said plurality of channels in said aiming assembly are configured to align a portion of said plurality of screws with said plurality of plate apertures in said first fixation plate;
said first fixation plate having
a first end,
a second end,
a top surface, and
a bottom surface;
said plurality of plate apertures comprising a first plate aperture and a second first plate aperture;
said channeling assembly having
a thickness,
a top surface, and
a bottom surface;
said bottom surface of said first fixation plate substantially couples with a surface of a first bone of a skeletal system;
said bottom surface said aiming assembly substantially couples with said top surface of said first fixation plate;
said plurality of channels configured to receive and align a portion of said plurality of screws as said plurality of screws are inserted through said plurality of plate apertures and secured to said first bone;
said first fixation plate being extramedullary attached to a portion of said surface of said first bone;
said plurality of channels of said aiming assembly being configured to align and triangulate
a one or more extramedullary locations including a portion of said plurality of plate apertures, and
a one or more intramedullary locations including said one or more threaded female apertures in said first intramedullary nail;
a portion of said plurality of channels of said aiming assembly being configured to receive a portion of said plurality of screws, align said portion of said plurality of screws with said one or more extramedullary locations and said one or more intramedullary locations;
a one or more apertures in said first intramedullary nail comprise a one or more threaded female apertures;
said one or more threaded female apertures comprise a first female aperture;
said aiming assembly comprising a triangulating screw guide;
said triangulating screw guide comprising a plurality of channels;
said triangulating screw guide is configured to releaseably attach to said one or more plates and to align with portions of said first intramedullary nail; and
with said triangulating screw guide attached to said first intramedullary nail, said plurality of channels in said triangulating screw guide are aligned with said one or more threaded female apertures of said first intramedullary nail.

2. A surgical aiming system comprising:
a one or more fixation plates, an aiming assembly, a one or more nails and a plurality of screws;
said one or more fixation plates comprising a first fixation plate;
said one or more nails comprising a first intramedullary nail;
said first intramedullary nail comprising a one or more apertures comprising a first aperture;
said aiming assembly comprising a channeling assembly having a plurality of channels;
said one or more fixation plates comprising a plurality of plate apertures configured to receive a portion of said plurality of screws;
said plurality of channels in said aiming assembly are configured to align a portion of said plurality of screws with said plurality of plate apertures in said first fixation plate;
said first fixation plate having
a first end,
a second end,
a top surface, and
a bottom surface;
said plurality of plate apertures comprising a first plate aperture and a second first plate aperture;
said channeling assembly having
a thickness,
a top surface, and
a bottom surface;
said bottom surface of said first fixation plate substantially couples with a surface of a first bone of a skeletal system;
said bottom surface said aiming assembly substantially couples with said top surface of said first fixation plate;
said plurality of channels configured to receive and align a portion of said plurality of screws as said plurality of screws are inserted through said plurality of plate apertures and secured to said first bone;
said first fixation plate being extramedullary attached to a portion of said surface of said first bone;
said plurality of channels of said aiming assembly being configured to align and triangulate
a one or more extramedullary locations including a portion of said plurality of plate apertures, and
a one or more intramedullary locations including said one or more apertures in said first intramedullary nail; and wherein,
a portion of said plurality of channels of said aiming assembly being configured to receive a portion of said plurality of screws, align said portion of said plurality of screws with said one or more extramedullary locations and said one or more intramedullary locations.

3. The surgical aiming system of claim 2 wherein:
said aiming assembly comprising
said channeling assembly, and
an intramedullary rod guide having a pointing device;
said intramedullary rod guide configured to selectively attach to said channeling assembly;
with said intramedullary rod guide attached to said channeling assembly, said pointing device is configured to point to an entry point in said first bone;
said entry point being a calculated as the ideal location to insert said first intramedullary nail into said first bone.

4. The surgical aiming system of claim 3 wherein:
said intramedullary rod guide having an upper portion and a lower portion;
said upper portion comprising a first channel and a second channel of said intramedullary rod guide;
said lower portion comprising said pointing device; and
said upper portion being configured to releaseably attach to said channeling assembly of said aiming assembly.

5. The surgical aiming system of claim 4 wherein:
said upper portion being substantially parallel with said channeling assembly of said aiming assembly when attached thereto; and
said lower portion bending down and away from said upper portion until said pointing device is substantially perpendicular to said upper portion.

6. The surgical aiming system of claim 4 wherein:
said entry point is configured such that when said first intramedullary nail will be inserted into said first bone without hitting a portion of said plurality of screws having been previously inserted into said first bone through a portion of said aiming assembly.

7. The surgical aiming system of claim 4 wherein:
a one or more apertures in said first intramedullary nail comprise a one or more threaded female apertures;
said one or more threaded female apertures comprise a first female aperture;
said aiming assembly comprising a triangulating screw guide;
said triangulating screw guide comprising a plurality of channels;
said triangulating screw guide is configured to releaseably attach to said one or more plates and to align with portions of said first intramedullary nail; and
with said triangulating screw guide attached to said first intramedullary nail, said plurality of channels in said triangulating screw guide are aligned with said one or more threaded female apertures of said first intramedullary nail.

8. The surgical aiming system of claim 2 wherein:
said first bone of said skeletal system belongs to a mammal.

9. The surgical aiming system of claim 2 wherein:
said first bone of said skeletal system belongs to a human.

10. The surgical aiming system of claim 2 further comprising:
said one or more plates comprising of said first fixation plate and a second fixation plate;
said one or more plates attach around a portion of said first bone;
said one or more plates each comprise a portion of said plurality of plate apertures;
said aiming assembly releaseably attaches to said one or more plates;
said plurality of channels in said aiming assembly align and direct said plurality of screws into and through said plurality of plate apertures in said one or more plates; and
said one or more plates hold a portion of said first bone from a plurality of surface locations around said one or more nails within said first bone.

11. The surgical aiming system of claim 2 wherein:
said alignment and entry points of said plurality of screws through said plurality of channels of said aiming assembly are predetermined to
avoid intersecting with said one or more nails or
to attach to a portion of said one or more nails.

12. The surgical aiming system of claim 2 wherein:
said channeling assembly is configured to selectively attach to said first intramedullary nail;
said first intramedullary nail comprising said plurality of apertures for engaging said plurality of screws;
said first fixation plate having a plurality of plate apertures for accepting said plurality of screws;
said channeling assembly is configured to guide plurality of screws into said first bone and through said plurality of apertures in said first intramedullary nail; and
said channeling assembly is configured such that at a first screw, among said plurality of screws, is guided into a first aperture in said first fixation plate and through said first female aperture in the first intramedullary nail.

13. The surgical aiming system of claim 12 wherein:
said plurality of screws comprise a second screw;
said one or more threaded female apertures in said first intramedullary nail comprise a second female aperture;
said plurality of plate apertures comprise a second plate aperture;
said channeling assembly is configured such that said second screw is guided through said second plate aperture in said first fixation plate and through said second female aperture in said first intramedullary nail.

14. A surgical aiming system installation method comprising:
attaching a first fixation plate with a second end of a first bone,
selectively attaching a channeling assembly having a pointing device to said first fixation plate,
triangulating an entry point on said first bone with said pointing device,
inserting a first intramedullary nail into said first bone at said entry point,
triangulating a one or more extramedullary locations on said first fixation plate with a one or more intramedullary locations on said first intramedullary nail with said channeling assembly,
inserting a plurality of screws into said first bone through said first fixation plate and said first intramedullary nail using said aiming assembly to align said plurality of screws; wherein,
said surgical aiming system comprising a one or more fixation plates, an aiming assembly, a one or more nails and a plurality of screws;
said first fixation plate comprising a portion of said one or more fixation plates
said aiming assembly comprising a plurality of channels configured for triangulating said one or more extramedullary locations with one or more intramedullary locations when said aiming assembly is attached to said first fixation plate;
said first intramedullary nail comprising a portion of said one or more nails and a plurality of screws;
said one or more fixation plates comprising said first fixation plate;
said first intramedullary nail comprising a one or more apertures comprising a first aperture;
said aiming assembly comprising a channeling assembly having a plurality of channels;
said one or more fixation plates comprising a plurality of plate apertures configured to receive a portion of said plurality of screws;
said plurality of channels in said aiming assembly are configured to align a portion of said plurality of screws with said plurality of plate apertures in said first fixation plate;
said first fixation plate having
a first end,
a second end,
a top surface, and
a bottom surface;
said plurality of plate apertures comprising a first plate aperture and a second first plate aperture;
said channeling assembly having
a thickness,
a top surface, and
a bottom surface;
said bottom surface of said first fixation plate substantially couples with a surface of a first bone of a skeletal system;
said bottom surface said aiming assembly substantially couples with said top surface of said first fixation plate;
said plurality of channels configured to receive and align a portion of said plurality of screws as said plurality of screws are inserted through said plurality of plate apertures and secured to said first bone;
said first fixation plate being extramedullary attached to a portion of said surface of said first bone;
said plurality of channels of said aiming assembly being configured to align and triangulate
a one or more extramedullary locations including a portion of said plurality of plate apertures, and
a one or more intramedullary locations including said one or more apertures in said first intramedullary nail; and wherein,
a portion of said plurality of channels of said aiming assembly being configured to receive a portion of said plurality of screws, align said portion of said plurality of screws with said one or more extramedullary locations and said one or more intramedullary locations.

15. A surgical aiming system installation method of claim 14 further comprising:
stacking a channeling assembly on said first fixation plate;
stacking an intramedullary rod guide on top of said channeling assembly;
aligning a portion of a plurality of channels in said intramedullary rod guide and said channeling assembly and said first fixation plate;
guiding said plurality of screws through said plurality of channels; and
attaching said plurality of screws to said first bone.

16. A surgical aiming system installation method of claim 15 further comprising:
marking an entry point with a pointing device of said intramedullary rod guide; and
drilling a portal at said entry point for inserting a first intramedullary nail into a medullary cavity.

17. A surgical aiming system installation method of claim 13 further comprising:
stacking a channeling assembly on said first fixation plate;
attaching a triangulating screw guide to said channeling assembly;
inserting a first intramedullary nail through a portion of said triangulating screw guide; and
attaching said first intramedullary nail on a portion of said triangulating screw guide.

18. A surgical aiming system installation method of claim 17 further comprising:
inserting a portion of said plurality of screws through a plurality of channels of said triangulating screw guide;
locating a one or more threaded female apertures with said portion of said plurality of screws; and
securing said first fixation plate and said first intramedullary nail to one another in a fixed position.

19. A surgical aiming system installation method of claim 17 further comprising:
inserting a one or more tissue sleeve into said one or more apertures;
pressing said tissue sleeve through a tissue of a patient;
inserting a portion of a drill sleeve through said one or more tissue sleeve;
pressing said drill sleeve into patient;
inserting a portion of a trocar through said drill sleeve down to said first bone;
removing said trocar;
drilling through said drill sleeve;
removing said drill sleeve from said one or more tissue sleeve; and
screwing a one or more screws through said one or more tissue sleeve and into a one or more threaded female apertures of said first intramedullary nail.

20. A surgical aiming system installation method of claim 17 further comprising:
aligning a second fixation plate with a styloid portion of said first bone;
aligning said channeling assembly and said triangulating screw guide with said first fixation plate; and
attaching said second fixation plate to said first fixation plate.
